(19) United States
(12) Patent Application Publication
(10) Pub. No.: US 2025/0186718 A1
(43) Pub. Date: Jun. 12, 2025

(54) DRUG DELIVERY DEVICE WITH ELECTRONICS

(71) Applicant: Norton (Waterford) Limited, Waterford (IE)

(72) Inventors: Tai Chan, Cambridge, MA (US); Enrique Calderon Oliveras, Barcelona (ES)

(73) Assignee: Norton (Waterford) Limited, Waterford (IE)

(21) Appl. No.: 18/843,847

(22) PCT Filed: Mar. 17, 2023
(86) PCT No.: PCT/EP2023/056923

§ 371 Date: Sep. 4, 2024

Related U.S. Application Data
(60) Provisional application No. 63/321,195, filed on Mar. 18, 2022.

(51) Int. Cl. A61M 15/00

(52) U.S. Cl. CPC A61M 15/008; A61M 2205/3553

(57) ABSTRACT

A system may include an inhaler (100) and an external device (904). The inhaler may include a main body (104) having a mouthpiece (106), medicament, and an electronics module (120). The electronics module may include a processor (1026), memory (1030), a sensor (1027), a switch (130), and a wireless communication circuit (129). The processor may generate a dose record in response to a dosing event. The dosing event may be an actuation of the switch (130) or measurements received by the sensor (1027). The processor (1026) may store the first dose record in memory and cause the wireless communication circuit (129) to transmit advertising data. The advertising data may include a dose record indicator that indicates that one or more dose records are stored in the memory (1030) of the electronics module (120). The external device (904) may receive the advertising data and determine, based on the advertising data, whether the inhaler (100) includes records not stored on the external device.

### CROSS-REFERENCE TO RELATED APPLICATIONS

This application claims the benefit of Provisional U.S. Patent Application No. 63/321,195, filed Mar. 18, 2022, the disclosure of which is incorporated herein by reference in its entirety.

### BACKGROUND

Drug delivery devices facilitate the delivery of medication into a patient's body via various routes of administration. Typical routes of administration include oral, topical, sublingual inhalation, injection and the like. The devices may be used to deliver medications for the treatment various diseases, ailments and medical conditions. Inhalation devices, for example, may be used to treat asthma, chronic obstructive pulmonary disease (COPD) and cystic fibrosis (CF). While drug delivery devices are designed to deliver an appropriate dose of medication to a patient as part of a therapeutic treatment, the effectiveness of a particular treatment may be influenced by non-physiological factors, such as the patient's adherence and compliance.

In the context of a drug therapy, adherence may refer to the degree to which a patient is following a prescribed dosing regimen. For example, if the patient's prescription calls for two doses each day, and the patient is taking two doses per day, the patient may be considered 100% adherent. If the patient is only taking one dose per day, he or she may be deemed only 50% adherent. In the latter case, the patient may not be receiving the treatment prescribed by his or her doctor, which may negatively affect the efficacy of the therapeutic treatment.

Compliance may refer to a patient's technique when using a particular drug delivery device. If the patient is using the device in a manner that is recommended by a doctor or by a manufacturer, the device is likely to deliver the desired dose of medication and the patient may be deemed compliant. However, if the device is not being used properly during drug administration, the device's ability to deliver a proper dose of medication may be compromised. As such, the patient may be deemed non-compliant. In the case of an inhalation device, for example, the patient may need to achieve a minimum inspiratory effort to ensure a full dose of medication is delivered from the device into the patient's lungs. For some patients, such as children and the elderly, meeting the requirements for full compliance may be difficult due to physical limitations, such as limited lung function. Accordingly, like adherence, failing to achieve full compliance may reduce the effectiveness of a prescribed treatment.

A patient's ability to achieve full compliance may be further complicated by certain physical properties of the medication. For example, some respiratory medications may consist of fine particles and/or may lack any odor or taste. Thus, a patient using an inhalation device may not be able to correct a non-compliant use because he or she may not be able to immediately detect or sense that medication is being inhaled and/or know whether the amount of inhaled medication complies with the prescription.

### SUMMARY

A drug delivery device may be adapted to include an electronics module that is configured to sense, track, and/or process usage conditions and parameters associated with the device (e.g., to improve adherence and compliance). The electronics module may be further configured to communicate the conditions and parameters to external devices, such as a smartphone, for similar and/or further processing. The inclusion of an electronics module in a drug delivery device opens the doors to a wealth of digital improvements and features to enhance the use of the device. The electronics module, in this context, may create a platform to leverage helpful smartphone applications and powerful data analytics. However, the introduction of electronics into any drug delivery device may introduce certain technical challenges, such as durability, reliability, electro-mechanical integration, power management, and drug delivery performance. The present disclosure provides solutions for inclusion of certain electrical components with a drug delivery device, such as an inhaler.

Examples of inhalation devices (e.g., breath-actuated and/or metered dose inhalers) and system that include inhalation devices are provided herein. A system may include an inhaler and an external device (e.g., a computer-readable storage medium residing on the external device). The inhaler may include a main body having a mouthpiece. The inhaler may include medicament. The inhaler may include an electronics module comprising a processor, memory, a sensor, a switch, and a wireless communication circuit. In some examples, the communication circuit may include a Bluetooth communication circuit. The processor of the electronics module may be configured to generate a dose record in response to a dosing event (e.g., a new dose record). The dosing event may be an actuation of the switch or measurements received by the sensor (e.g., measurements exceeding a threshold that is indicative of a user's inhalation through the inhaler). The processor of the electronics module may be configured to store the first dose record in memory, and cause the wireless communication circuit to transmit advertising data. The advertising data may include a dose record indicator that indicates that one or more dose records are stored in the memory of the electronics module. The one or more dose records may include the dose record (e.g., the new dose record).

The computer-readable storage medium may include executable instructions that, when executed by a processor of an external device, cause the processor of the external device to determine, based on the dose record indicator, that the first dose record has not previously been transmitted by the electronics module, and determine to enter a connected state with the communication circuit of the electronics module in response to the determination that the first dose record has not previously been transmitted by the electronics module. In some examples, the computer-readable storage medium may be further configured to cause the processor of the external device to determine to not enter the connected state with the communication circuit of the electronics module in response to a determination that the advertising data indicates that the one or more dose records stored in the memory of the electronics module were previously transmitted by the electronics module (e.g., either directly to the external device or to the external device via a server). In some examples, the computer-readable storage medium may be further configured to cause the processor of the external device to determine to not enter the connected state with the communication circuit of the electronics module in response to the dose record indicator missing from the advertising data.

In some examples, the dose record indicator may include an indication of a total number of dose records stored in the memory. In some examples, the dose record indicator may include an indication of a present dose count of the inhaler. In some examples, the dose record indicator may include an indication of the number of dosing events stored in the memory since the last time the inhaler entered a connected state with an external device.

The processor of the electronics module may be configured to receive a scan request from the external device, and cause the communication circuit to transmit a scan response message, wherein the scan response message comprises the last two dose records generated by the processor of the electronics module. The scan response message may include an inhalation start time, an inhalation duration, an inhalation peak, a time-to-peak, and an inhalation volume. The scan response message may include a switch actuation start time and a switch actuation close time. The computer-readable storage medium may be configured to cause the processor of the external device to send the scan request to the communication circuit of the electronics module in response to the determination that the one or more dose records have not been previously transmitted by the electronics module, and determine to enter a connected state with the communication circuit of the electronics module in response to a determination that the last two dose records included in the scan response message have not been previously transmitted by the electronics module. In some examples, the advertising data may include a data available indication that indicates that data is available from the inhalation device and is ready for wireless download.

In some examples, the inhaler further comprises a mouthpiece cover, and where movement of the mouthpiece cover from a closed position to an open position is configured to cause the switch to be actuated. The processor of the electronics module may be configured to generate the one or more dose records in response to measurements received from the sensor exceeding a threshold indicative of a user's inhalation via the mouthpiece of the inhaler.

The computer-readable storage medium may be configured to cause the processor of the external device to determine that the first dose record has not been previously transmitted by the electronics module to the external device when the first dose record has not been transmitted to the external device or any other device. The computer-readable storage medium may be configured to cause the processor of the external device to determine that the first dose record has not been previously transmitted by the electronics module to the external device when the first dose record has been transmitted to another external device.

The computer-readable storage medium may be configured to cause the processor of the external device to receive the first dose record from a server, and determine to not enter the connected state with the communication circuit of the electronics module in response to a determination that the first dose record indicated by the advertising data was received by the external device from the server. The computer-readable storage medium may be configured to cause the processor of the external device to receive a second dose record of the inhalation device from a server, and determine to not enter the connected state with the communication circuit of the electronics module in response a reception of advertising data from the inhalation device that includes a second dose record indicator that indicates the second dose record.

An inhaler may be configured to enter a fast advertising mode following a failed data transfer. An inhaler may include a main body having a mouthpiece, medicament, and an electronics module comprising a processor, a sensor, a switch, and a wireless communication circuit. The processor may be configured to generate a dose record in response to a dosing event, store the dose record in memory, and cause the communication circuit to transmit advertising data at a first advertising rate for an advertising period. The dosing event comprises one or more of actuation of the switch or measurements received from the sensor, and the advertising data comprises a dose record indicator that indicates that one or more dose records are stored in the memory of the electronics module. The processor may be configured to determine that a transfer of data between the electronics module and an external device has failed, and cause the communication circuit to transmit advertising data at the first advertising rate in response to the determination that the transfer of data between the electronics module and an external device has failed. The processor may be configured to cause the communication circuit to transmit advertising data at a second advertising rate in response the elapse of the advertising period. In some examples, the first advertising rate may be once every 100 ms, and the second advertising rate may be once every 10 seconds.

The processor may be configured to cause the wireless communication circuit to attempt to transmit the one or more dose records to the external device when the wireless communication circuit is in the connected state with the external device. The processor may be configured to determine that the transfer of data between the electronics module and the external device has failed while the wireless communication circuit was in a connected state with the external device. In some examples, when the electronics module is in a sleep state, the processor is configured to cause the wireless communication circuit to transmit the advertising data at the second advertising rate.

The processor may be configured to cause the wireless communication circuit to transmit the advertising data at the first advertising rate for the first predetermined period of time after a detection of an actuation of the switch. For example, the processor may be configured to cause the communication circuit to transmit the advertising data at the second advertising rate in response to a determination that transmission of the one or more dose records to the external device was successful.

In some examples, the inhaler may include a mouthpiece cover, and movement of the mouthpiece cover of the inhaler from an open position to a closed position may be configured to actuate the switch.

In some examples, the processor may be configured to generate a dose record in response to a dosing event, and store the dose record in memory, wherein the dosing event comprising actuation of the switch or measurements received from the sensor. The processor may also be configured to set a retry value and an advertising period timer in response the dosing event, decrease the retry value in response to a failed data transmission or in response to the expiration of the advertising period timer, and reset the advertising period timer based on a determination that the retry value is greater than a retry threshold. The processor may be configured to cause the communication circuit to transmit advertising data at a first advertising rate during the pendency of the advertising period timer, and cause the communication circuit to transmit advertising data at a second advertising rate upon the expiration of the advertising period timer and a determination that the retry value is less than or equal to the retry threshold. In some instances, the first advertising rate may be once every 100 ms, and the second advertising rate may be once every 10 seconds. Note that, in an alternative implementation, retry value may count up instead of counting down. That is, the processor may be configured to increase the retry value in response to a failed data transmission or in response to the expiration of the advertising period timer, and reset the advertising period timer based on a determination that the retry value is less than a retry threshold. The processor may be configured to cause the communication circuit to transmit advertising data at a second advertising rate upon the expiration of the advertising period timer and a determination that the retry value is greater than or equal to the retry threshold.

The advertising data may include a dose record indicator that indicates that one or more dose records are stored in the memory of the electronics module. In some examples, the advertising period timer is 60 seconds, and the retry threshold is 3. When the electronics module is in a sleep state, the processor is configured to cause the wireless communication circuit to transmit advertising data at the second advertising rate. The processor may be configured to set the retry value and the advertising period timer in response to a detection of an actuation of the switch. For example, the inhaler may include a mouthpiece cover and movement of the mouthpiece cover of the inhaler from an open position to a closed position may be configured to actuate the switch.

An inhaler may include a rechargeable battery and may be configured to enter a low battery mode when the battery power drops below a threshold. For instance, the inhaler may include a main body having a mouthpiece, medicament, and an electronics module comprising a processor, a battery, a switch, and a wireless communication circuit. The processor may be configured to determine whether an energy level (e.g., power level, charge level, voltage, etc.) of the battery is below a threshold. The processor may be configured to transition the electronics module from a sleep state to an active state in response to a closing of the switch and a determination that the energy level of the battery is not below the threshold. The processor may be configured to transition the electronics module from the sleep state to a low power active state in response to a closing of the switch and a determination that the energy level of the battery is below the threshold. The electronics module may be configured to use more battery power when in the active state than when in the sleep state, use more battery power when in the active state than when in the low power active state, and use more battery power when in the low power active state than when in the sleep state. The wireless communication circuit may be configured to transmit advertising data to an external device at a first advertising rate in the active state. When in the low power active state, the processor may be configured to store a dose record in memory in response to a dosing event and cause the wireless communication circuit to not transmit advertising data or to transmit advertising data to the external device at a second advertising rate. The second advertising rate may be less than (e.g., slower than) the first advertising rate. The dosing event may include one or more of actuation of the switch or measurements received from the sensor.

The processor may be configured to prevent the wireless communication circuit from transmitting the dose record to the external device when the energy level of the battery is below the threshold. For example, the processor may be configured to cause the wireless communication circuit to transmit the dose record to the external device upon a determination that the energy level of the battery increased above the threshold. The first advertising rate may be once every 100 ms, and the second advertising rate may be once every 10 seconds.

When causing the wireless communication circuit to transmit the advertising data to the external device at the second advertising rate, the processor may be configured to cause the wireless communication circuit to transmit the advertising data to the external device at a slow advertising rate for a predetermined period of time after the generation of a dose record, and thereafter, cause the wireless communication circuit to cease transmission of the advertising data until generation of another dose record.

The threshold may be a first threshold (e.g., of a plurality of thresholds), and the processor may be configured to determine when the energy level of the battery is below a second threshold that is less than the first threshold. When the energy level is below the first threshold and above the second threshold, the processor may be configured to cause the wireless communication circuit to transmit the advertising data at the second advertising rate. When the energy level is below the second threshold, the processor may be configured to cause the wireless communication to not transmit the advertising data.

When the electronics module is in the active state, the processor may be configured to generate the dose record in response to the dosing event, and cause the wireless communication circuit to transmit advertising data to an external device. The dosing event may include actuation of the switch. When the electronics module is in the low power active sate, the processor may be configured to generate the dose record in response to the dosing event, and configured to not cause the wireless communication circuit to transmit the advertising data to the external device. For example, the electronics module may be configured to not power the wireless communication circuit when the electronics module is in the low power active state.

In some examples, the electronics module may include a sensor. When the electronics module is in the active state, the processor may be configured to receive measurements from the sensor, generate a dose record in response to a dosing event, and cause the wireless communication circuit to transmit the dose record to an external device. When the electronics module is in the low power active sate, the processor may be configured to receive the measurements from the sensor, generate the dose record based on the measurements received from the sensor, and not cause the communication circuit to transmit the dose record to the external device.

The processor may be configured to receive a battery voltage feedback signal, where the battery voltage feedback signal may indicate the energy level of battery. For example, the energy level of the battery may include a voltage level of the battery.

In some examples, the processor may be configured to determine whether an energy level of the battery is below a threshold. The processor may be configured to store a dose record in memory in response to a dosing event and cause the wireless communication circuit to transmit advertising data to an external device when the energy level of the battery is not below the threshold. The processor may be configured to store a dose record in memory in response to a dosing event and not cause the wireless communication circuit to transmit advertising data to the external device when the energy level of the battery is below the threshold. For example, the processor may be configured to transition the electronics module to a low power active state in response to a determination that the energy level of the battery is below the threshold, and may be configured to transition the electronics module to an active state in response to a determination that the energy level of the battery is not below the threshold. Further, and for example, the processor may be configured to cause the wireless communication circuit to transmit the dose record to the external device upon a determination that the energy level of the battery increased above the threshold.

In some examples, the processor may be configured to determine whether an energy level of the battery is below a threshold. The processor may be configured to store a dose record in memory in response to a dosing event and cause the wireless communication circuit to transmit advertising data to an external device at a first advertising rate when the energy level of the battery is not below the threshold. The processor may be configured to store a dose record in memory in response to a dosing event and cause the wireless communication circuit to transmit advertising data to the external device at a second advertising rate when the energy level of the battery is below the threshold, where the second advertising rate may be less than the first advertising rate.

### BRIEF DESCRIPTION OF THE DRAWINGS

FIG. 1 is a front perspective view of an example inhalation device.

FIG. 2 is a cross-sectional interior perspective view of the example inhalation device of FIG. 1.

FIG. 3 is an exploded perspective view of the example inhalation device of FIG. 1 with a top cap removed to expose an electronics module.

FIG. 4 is an exploded perspective view of the top cap and the electronics module of the example inhalation device of FIG. 1.

FIG. 5A is a partial cross-sectional view of the example inhalation device of FIG. 1 with a mouthpiece cover of the inhalation device in a closed position.

FIG. 5B is a partial cross-sectional view of the example inhalation device of FIG. 1 with the mouthpiece cover of the inhalation device in a partially open position.

FIG. 5C is a partial cross-sectional view of the example inhalation device of FIG. 1 with the mouthpiece cover of the inhalation device in a partially open position.

FIG. 5D is a partial cross-sectional view of the example inhalation device of FIG. 1 with the mouthpiece cover of the inhalation device in a fully open position.

FIGS. 6A and 6B include a flow diagram that illustrates an example process for transitioning between one or more power states and/or operational modes associated with the inhalation device.

FIG. 7 is a flow diagram that illustrates an example process for determining whether to connect with the inhalation device.

FIG. 8 is a flow diagram that illustrates an example process for sending dose records to an external device.

FIG. 9 is a flow diagram that illustrates an example process for operation of an inhalation device in a low power active state.

FIG. 10 is a graph of exemplary airflow rates through the example inhalation device of FIG. 1 based on pressure measurements recorded by the electronics module.

FIG. 11 is a diagram of an example system including an inhalation device.

FIG. 12 is a block diagram of an example electronics module of the example inhalation device of FIG. 1.

FIG. 13 is a block diagram of an example external device.

### DETAILED DESCRIPTION

The present disclosure describes devices, systems and methods for sensing, tracking and/or processing usage conditions and parameters associated with a drug delivery device. The devices, systems and methods are described in the context of a breath-actuated inhalation device for delivering medication into a user's lungs. However, the described solutions are equally applicable to other drug delivery devices, such as an injector, a metered-dose inhaler, a nebulizer, a transdermal patch, or an implantable.

Asthma and COPD are chronic inflammatory disease of the airways. They are both characterized by variable and recurring symptoms of airflow obstruction and bronchospasm. The symptoms include episodes of wheezing, coughing, chest tightness and shortness of breath. The symptoms are managed by avoiding triggers and by the use of medicaments, particularly inhaled medicaments. The medicaments include inhaled corticosteroids (ICSs) and bronchodilators.

Inhaled corticosteroids (ICSs) are steroid hormones used in the long-term control of respiratory disorders. They function by reducing the airway inflammation. Examples include budesonide, beclomethasone (dipropionate/dipropionate HFA), fluticasone (propionate), mometasone (furoate), ciclesonide and dexamethasone (sodium). Parentheses indicate examples (e.g., preferred) salt or ester forms.

Different classes of bronchodilators target different receptors in the airways. Two commonly used classes are β2-agonists and anticholinergics. β2-Adrenergic agonists (or “β2-agonists”) act upon the β2-adrenoceptors which induces smooth muscle relaxation, resulting in dilation of the bronchial passages. They tend to be categorised by duration of action. Examples of long-acting β2-agonists (LABAs) include formoterol (fumarate), salmeterol (xinafoate), indacaterol (maleate), bambuterol (hydrochloride), clenbuterol (hydrochloride), olodaterol (hydrochloride), carmoterol (hydrochloride), tulobuterol (hydrochloride) and vilanterol (triphenylacetate). Examples of short-acting β2-agonists (SABA) are albuterol (sulfate) and terbutaline (sulfate).

Typically, short-acting bronchodilators provide a rapid relief from acute bronchoconstriction (and are often called “rescue” or “reliever” medicines), whereas long-acting bronchodilators help control and prevent longer-term symptoms. However, some rapid-onset long-acting bronchodilators may be used as rescue medicines, such as formoterol (fumarate). Thus, a rescue medicine provides relief from acute bronchoconstriction. The rescue medicine is taken as-needed/prn (pro re nata). The rescue medicine may also be in the form of a combination product, e.g. ICS-formoterol (fumarate), typically budesonide-formoterol (fumarate) or beclomethasone (dipropionate)-formoterol (fumarate). Thus, the rescue medicine is preferably a SABA or a rapid-acting LABA, more preferably albuterol (sulfate) or formoterol (fumarate), and most preferably albuterol (sulfate).

Anticholinergics (or “antimuscarinics”) block the neurotransmitter acetylcholine by selectively blocking its receptor in nerve cells. On topical application, anticholinergics act predominantly on the M3 muscarinic receptors located in the airways to produce smooth muscle relaxation, thus producing a bronchodilatory effect. Examples of long-acting muscarinic antagonists (LAMAs) include tiotropium (bromide), oxitropium (bromide), aclidinium (bromide), umeclidinium (bromide), ipratropium (bromide), glycopyrronium (bromide), oxybutynin (hydrochloride or hydrobromide), tolterodine (tartrate), trospium (chloride), solifenacin (succinate), fesoterodine (fumarate) and darifenacin (hydrobromide).

A number of approaches have been taken in preparing and formulating these medicaments for delivery by inhalation, such as via a dry powder inhaler (DPI), a pressurized metered dose inhaler (pMDI) or a nebulizer. Although described primarily in the context of a DPI, the inhaler and inhalation devices described herein could take any form, such as MDIs, nebulizers, or other inhalation devices.

According to the GINA (Global Initiative for Asthma) Guidelines, a step-wise approach can be taken to the treatment of asthma. At step 1, which represents a mild form of asthma, the patient is given an as-needed SABA, such as albuterol sulfate. The patient may also be given an as-needed low-dose ICS-formoterol, or a low-dose ICS whenever the SABA is taken. At step 2, a regular low-dose ICS is given alongside the SABA, or an as-needed low-dose ICS-formoterol. At step 3, a LABA is added. At step 4, the doses are increased and at step 5, further add-on treatments are included such as an anticholinergic or a low-dose oral corticosteroid. Thus, the respective steps may be regarded as treatment regimens, which regimens are each configured according to the degree of acute severity of the respiratory disease.

COPD is a leading cause of death worldwide. It is a heterogeneous long-term disease comprising chronic bronchitis, emphysema and also involving the small airways. The pathological changes occurring in patients with COPD are predominantly localized to the airways, lung parenchyma and pulmonary vasculature. Phenotypically, these changes reduce the healthy ability of the lungs to absorb and expel gases.

Bronchitis is characterized by long-term inflammation of the bronchi. Common symptoms may include wheezing, shortness of breath, cough and expectoration of sputum, all of which are highly uncomfortable and detrimental to the patient's quality of life. Emphysema is also related to long-term bronchial inflammation, wherein the inflammatory response results in a breakdown of lung tissue and progressive narrowing of the airways. In time, the lung tissue loses its natural elasticity and becomes enlarged. As such, the efficacy with which gases are exchanged is reduced and respired air is often trapped within the lung. This results in localised hypoxia, and reduces the volume of oxygen being delivered into the patient's bloodstream, per inhalation. Patients therefore experience shortness of breath and instances of breathing difficulty.

Patients living with COPD experience a variety, if not all, of these symptoms on a daily basis. Symptom severity will be determined by a range of factors but most commonly will be correlated to the progression of the disease. These symptoms, independent of their severity, are indicative of stable COPD and this disease state is maintained and managed through the administration of a variety drugs. The treatments are variable, but often include inhaled bronchodilators, anticholinergic agents, long-acting and short-acting β2-agonists and corticosteroids. The medicaments are often administered as a single therapy or as combination treatments.

Patients are categorized by the severity of their COPD using categories defined in the GOLD Guidelines (Global Initiative for Chronic Obstructive Lung Disease, Inc.). The categories are labelled A-D and the recommended first choice of treatment varies by category. Patients in group A are recommended a short-acting muscarinic antagonist (SAMA) prn or a short-acting β2-aginist (SABA) prn. Patients in group B are recommended a long-acting muscarinic antagonist (LAMA) or a long-acting β2-aginist (LABA). Patients in group C are recommended an inhaled corticosteroid (ICS)+a LABA, or a LAMA. Patients in group D are recommended an ICS+a LABA and/or a LAMA.

Patients suffering from respiratory diseases like asthma or COPD suffer from periodic exacerbations beyond the baseline day-to-day variations in their condition. An exacerbation is an acute worsening of respiratory symptoms that require additional therapy, i.e. a therapy going beyond their maintenance therapy.

For asthma, the additional therapy for a moderate exacerbation are repeated doses of SABA, oral corticosteroids and/or controlled flow oxygen (the latter of which requires hospitalization). A severe exacerbation adds an anticholinergic (typically ipratropium bromide), nebulized SABA or IV magnesium sulfate.

For COPD, the additional therapy for a moderate exacerbation is repeated doses of SABA, oral corticosteroids and/or antibiotics. A severe exacerbation adds controlled flow oxygen and/or respiratory support (both of which require hospitalization). An exacerbation within the meaning of the present disclosure includes both moderate and severe exacerbations.

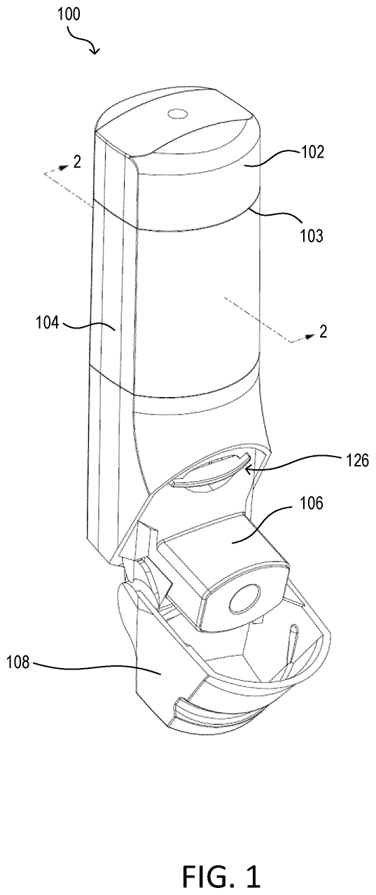

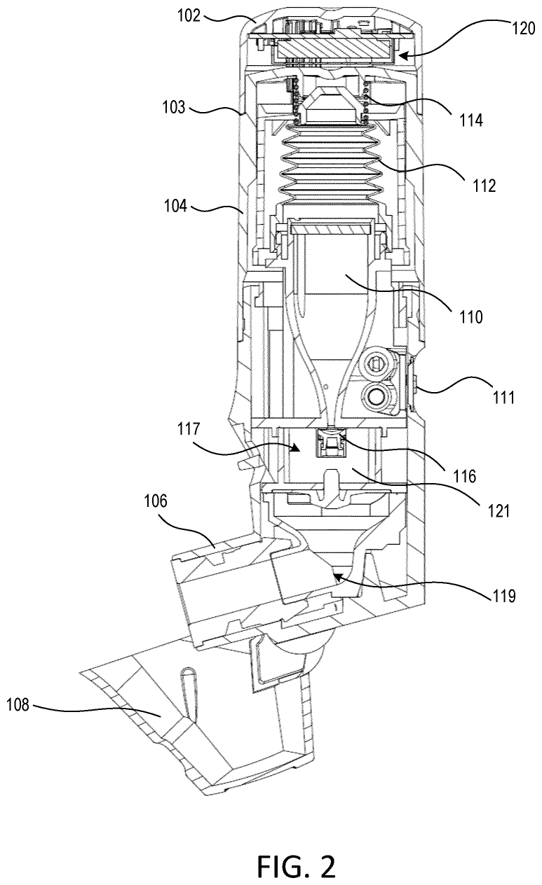

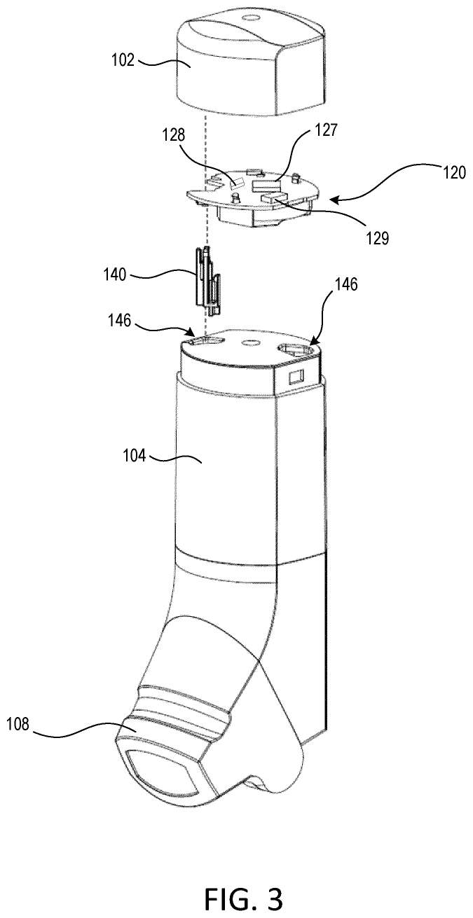

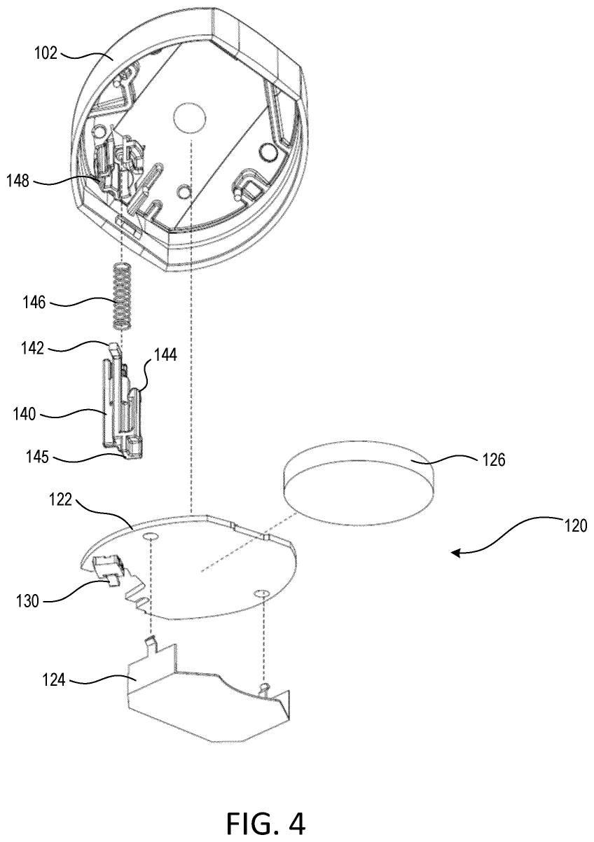

FIG. 1 is a front perspective view of an example inhalation device 100. FIG. 2 is a cross-sectional interior perspective view of the example inhalation device 100. FIG. 3 is an exploded perspective view of the example inhalation device 100 with a top cap removed to expose an electronics module. FIG. 4 is an exploded perspective view of the top cap and the electronics module of the example inhalation device 100.

The example, inhalation device 100 may be a breath-actuated inhalation device. The inhalation device 100 may include a top cap 102, a main housing 104, a mouthpiece 106, a mouthpiece cover 108, an electronics module 120, and an air vent 126. The mouthpiece cover 108 may be hinged to the main housing 104 so that it may open and close to expose the mouthpiece 106. Although illustrated as a hinged connection, the mouthpiece cover 106 may be connected to the inhalation device 100 through other types of connections. Moreover, while the electronics module 120 is illustrated as housed within the top cap 102 at the top of the main housing 104, the electronics module 120 may be integrated and/or housed within main body 104 of the inhalation device 100.

Inside the main housing 104, the inhalation device 100 may include a medication reservoir and a dose delivery mechanism/system. For example, the inhalation device 100 may include a medication reservoir 110 (e.g., a hopper), a bellows 112, a bellows spring 114, a yoke 118, a dosing cup 116, a dosing chamber 117, a deagglomerator 121 and a flow pathway 119. The medication reservoir 110 may include medication, such as dry powder mediation, for delivery to the user. When the mouthpiece cover 108 is moved to expose the mouthpiece 106 (e.g., from a closed position to an open position), the bellows 112 may compress to deliver a dose of medication from the medication reservoir 110 to the dosing cup 116. Thereafter, a user may inhale through the mouthpiece 106 in an effort to receive the dose of medication. The airflow generated from the user's inhalation may cause the deagglomerator 121 to aerosolize the dose of medication by breaking down the agglomerates of the medicament in the dose cup 116. The deagglomerator 121 may be configured to aerosolize the medication when the airflow through the flow pathway 119 meets or exceeds a particular rate, or is within a specific range. When aerosolized, the dose of medication may travel from the dosing cup 116, into the dosing chamber 117, through the flow pathway 119, and out of the mouthpiece 106 to the user. If the airflow through the flow pathway 119 does not meet or exceed a particular rate, or is not within a specific range, some or all of the medication may remain in the dosing cup 116. In the event that the medication in the dosing cup 116 has not been aerosolized by the deagglomerator 121, another dose of medication may not be delivered from the medication reservoir 110 when the mouthpiece cover 108 is subsequently opened. Thus, a single dose of medication may remain in the dosing cup until the dose has been aerosolized by the deagglomerator 121.

As the user inhales through the mouthpiece 106, air may enter the air vent 126 to provide a flow of air for delivery of the medication to the user. The flow pathway 119 may extend from the dosing chamber 117 to the end of the mouthpiece 106, and include the dosing chamber 117 and the internal portions of the mouthpiece 106. The dosing cup 116 may reside within or adjacent to the dosing chamber 117. Further, the inhalation device 100 may include a dose counter 111 that is configured to be initially set to a number of total doses of medication within the medication reservoir 110 and to decrease by one each time the mouthpiece cover 108 is moved from the closed position to the open position.

Although illustrated as a combination of the bellows 112, the bellows spring 114, the yoke 118, the dosing cup 116, the dosing chamber 117, and the deagglomerator 121, the dose delivery mechanism of the inhalation device 100 may include a subset of the components described and/or the inhalation device 100 may include a different dose delivery mechanism (e.g., based on the type of inhalation device, the type of medication, etc.). For instance, in some examples the medication may be included in a blister strip and the dose delivery mechanism (e.g., which may include one or more wheels, levers, and/or actuators) may be configured to advance the blister strip, open a new blister that includes a dose of medication, and make that dose of medication available to a dosing chamber and/or mouthpiece for inhalation by the user.

The top cap 102 may be attached to the main housing 104. For example, the top cap 102 may be attached to the main housing 104 through the use of one or more clips that engage recesses on the main housing 104. The top cap 102 may overlap a portion of the main housing 104 when connected, for example, such that a substantially pneumatic seal exists between the top cap 102 and the main housing 104. The top surface of the main housing 104 may include one or more (e.g., two) orifices 146. One of the orifices 146 may be configured to accept a slider 140. For example, when the top cap 102 is attached to the main housing 104, the slider 140 may protrude through the top surface of the main housing 104 via one of the orifices 146.

The slider 140 may define an arm 142, a stopper 144, and a distal base 145. The distal end 145 may be a bottom portion of the slider 140. The distal end 145 of the slider 140 may be configured to abut the yoke 118 that resides within the main housing 104 (e.g., and the mouthpiece cover 108 is in the closed or partially open position). The distal end 145 may be configured to abut a top surface of the yoke 118 when the yoke 118 is in any radial orientation. For example, the top surface of the yoke 118 may include a plurality of apertures (not shown), and the distal end 145 of the slider 140 may be configured to abut the top surface of the yoke 118, for example, whether or not one of the apertures is in alignment with the slider 140.

The top cap 102 may include a slider guide 148 that is configured to receive a slider spring 146 and the slider 140. The slider spring 146 may reside within the slider guide 148. The slider spring 146 may engage an inner surface of the top cap 102, and the slider spring 146 may engage (e.g., abut) an upper portion (e.g., a proximate end) of the slider 140. When the slider 140 is installed within the slider guide 148, the slider spring 146 may be partially compressed between the top of the slider 140 and the inner surface of the top cap 102. For example, the slider spring 146 may be configured such that the distal end 145 of the slider 140 remains in contact with the yoke 118 when the mouthpiece cover 108 is closed. The distal end 145 of the slider 145 may also remain in contact with the yoke 118 while the mouthpiece cover 108 is being opened or closed. The stopper 144 of the slider 140 may engage a stopper of the slider guide 148, for example, such that the slider 140 is retained within the slider guide 148 through the opening and closing of the mouthpiece cover 108, and vice versa. The stopper 144 and the slider guide 148 may be configured to limit the vertical (e.g., axial) travel of the slider 140. This limit may be less than the vertical travel of the yoke 118. Thus, as the mouthpiece cover 108 is moved to an open position, the yoke 118 may continue to move in a vertical direction towards the mouthpiece 106 but the stopper 144 may stop the vertical travel of the slider 140 such that the distal end 145 of the slider 140 may no longer be in contact with the yoke 118.

The electronics module 120 may include a printed circuit board (PCB) assembly 122, a switch 130, a power supply (e.g., a battery 126), and/or a battery holder 124. The PCB assembly 122 may include surface mounted components, such as a sensor system 128, a wireless communication circuit 129, the switch 130, and or one or more indicators (not shown), such as one or more light emitting diodes (LEDs). The switch 130 may be a electromechanical switch or a semiconductor switching circuit (e.g., a transistor, such as a field-effect transistor). The electronics module 120 may include a controller (e.g., a processor) and/or memory. The controller and/or memory may be physically distinct components of the PCB 122. Alternatively, the controller and memory may be part of another chipset mounted on the PCB 122. For example, the wireless communication circuit 129 may include the controller and/or memory for the electronics module 120. The controller 127 of the electronics module 120 may include a microcontroller, a programmable logic device (PLD), a microprocessor, an application specific integrated circuit (ASIC), a field-programmable gate array (FPGA), or any suitable processing device or control circuit.

The controller 127 may access information from, and store data in the memory. The memory may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory may be internal to the controller 127. The controller 127 may also access data from, and store data in, memory that is not physically located within the electronics module 120, such as on a server or a smartphone.

The memory may comprise a computer-readable storage media or machine-readable storage media that maintains computer-executable instructions for performing one or more as described herein. For example, the memory may comprise computer-executable instructions or machine-readable instructions that include one or more portions of the procedures described herein. The controller 127 of the electronics module 120 may access the instructions from memory for being executed to cause the controller 127 of the electronics module 120 to operate as described herein, or to operate one or more other devices as described herein. The memory may comprise computer-executable instructions for executing configuration software. For example, the computer-executable instructions may be executed to perform, in part and/or in their entirety, one or more procedures, such as the procedures 200, 400, and/or 500 as described herein. Further, the memory may have stored thereon one or more settings and/or control parameters associated with the electronics module 120.

The sensor system 128 may include one or more sensors, such as one or more pressure sensors, temperature sensors, humidity sensors, acoustic sensors, optical sensors, orientation sensors, and/or the like. The pressure sensor(s) may include a barometric pressure sensor (e.g., an atmospheric pressure sensor), a differential pressure sensor, an absolute pressure sensor, and/or the like. The sensors may employ microelectromechanical systems (MEMS) and/or nanoelectromechanical systems (NEMS) technology. The pressure sensor(s) may be configured to provide an instantaneous pressure reading to the processor of the electronics module 120 and/or aggregated pressure readings over time. The pressure sensor(s) may be configured to measure a plurality of atmospheric pressures within the inhalation device 100. Examples of the sensors are described in reference to US 2020/0360630 A1, the entire disclosure of which are incorporated herein by reference. Further, it should be appreciated that the controller 127 of the electronics module 120 may be configured to convert pressure measurements received from the pressure sensor to a flow rate based on Bernoulli's principle (e.g., the Bernoulli/Venturi effect).

It will be appreciated that the atmospheric pressure within the device 100 (e.g., within the top cap 102 or within the housing 104) may be the same as or similar to the atmospheric pressure outside the device 100 when the device 100 is not being used. However, when a user inhales from the mouthpiece 106, the user's inhalation may cause the atmospheric pressure within the device 100 to decrease. Conversely, an exhalation into the mouthpiece 106 may cause the atmospheric pressure within the device 100 to increase. In both cases, the atmospheric pressure within the device 100 may differ from the atmospheric pressure outside of the device 100. Accordingly, certain parameters or metrics associated with the inhalation or exhalation may be determined by comparing changes in atmospheric pressure resulting from the inhalation or exhalation.

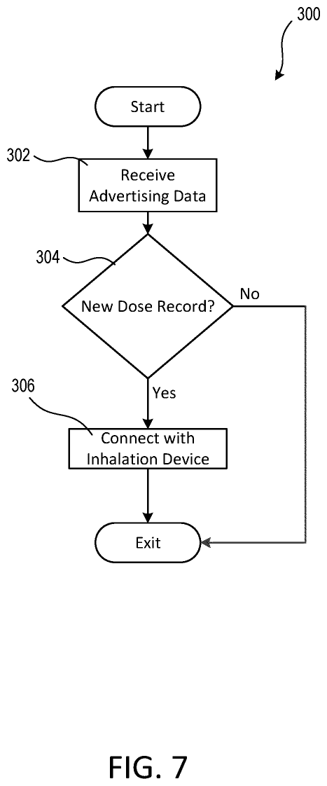

The controller 127 of the electronics module 120 may receive signals corresponding to measurements from the sensor system 128. The electronics module 120 (e.g., and/or a mobile application residing on an external device) may use measurements from the sensor system 128 to determine one or more inhalation events. The controller 127 may calculate or determine one or more airflow metrics (e.g., a peak flow rate, a time to peak flow rate, an inhaled volume, an inhalation duration, etc.) using the signals received from the sensor system 128. The airflow metrics may be indicative of a profile of airflow through the flow pathway 119 of the inhalation device 100. For example, if the sensor system 128 records a change in pressure of 0.3 kilopascals (kPA), the electronics module 120 may determine that the change corresponds to an airflow rate of approximately 45 liters per minute (Lpm) through the flow pathway 119. FIG. 7 shows an example of airflow rates based on various pressure measurements. It will be appreciated that the airflow rates and profile shown in FIG. 7 are merely examples and that determined rates may depend on the size, shape, and design of the inhalation device 100 and its internal components.

The airflow metrics may include one or more of an average flow of an inhalation/exhalation, a peak flow of an inhalation/exhalation (e.g., a maximum inhalation achieved), a volume of an inhalation/exhalation, a time to peak of an inhalation/exhalation, and/or the duration of an inhalation/exhalation. The airflow metrics may also be indicative of the direction of flow through the flow pathway 119. That is, a negative change in pressure may correspond to an inhalation from the mouthpiece 106, while a positive change in pressure may correspond to an exhalation into the mouthpiece 106. When calculating the airflow metrics, the electronics module 120 may be configured to eliminate or minimize any distortions caused by environmental conditions. For example, the electronics module 120 may “zero out” to account for changes in atmospheric pressure before and/or after calculating the airflow metrics. The one or more pressure measurements and/or airflow metrics may be timestamped and stored in the memory of the electronics module 120.

The controller 127 of the electronics module 120 may compare signals received from the sensor system 128 and/or the determined airflow metrics to one or more thresholds or ranges, for example, as part of an assessment of how the inhalation device 100 is being used and/or whether the use is likely to result in the delivery of a full dose of medication. For example, where the determined airflow metric corresponds to an inhalation with an airflow rate below a particular threshold, the electronics module 120 may determine that there has been no inhalation or an insufficient inhalation from the mouthpiece 106 of the inhalation device 100. If the determined airflow metric corresponds to an inhalation with an airflow rate above a particular threshold, the electronics module 120 may determine that there has been an excessive inhalation from the mouthpiece 106. If the determined airflow metric corresponds to an inhalation with an airflow rate within a particular range, the electronics module 120 may determine that the inhalation is “good”, or likely to result in a full dose of medication being delivered. As noted above, the electronics module 120 may include indicators, such as an LED. The indicators may be configured to provide feedback to users regarding their use of the inhalation device 100. Thus, in one example, an LED may illuminate or change color if the airflow metrics correspond to a good inhalation or to no inhalation. The airflow metrics may be computed and/or assessed via external devices as well (e.g., partially or entirely).

More specifically, the wireless communication circuit 129 in the electronics module 120 may include a transmitter and/or receiver (e.g., a transceiver), as well as additional circuitry. For example, the wireless communication circuit 129 may include a Bluetooth chip set (e.g., a Bluetooth Low Energy chip set), a ZigBee chipset, a Thread chipset, etc. As such, the electronics module 120 may wirelessly provide data such as pressure measurements, airflow metrics and/or other conditions related to usage of the inhalation device 100, to an external device, including a smartphone. The external device may include software for processing the received information and for providing compliance and adherence feedback to users of the inhalation device 100 via a graphical user interface (GUI).

The battery 126 may provide power to the components of the PCB 122. The battery 126 may be any suitable source for powering the electronics module 120, such as a coin cell battery, for example. The battery 126 may be rechargeable or non-rechargeable. The battery 126 may be housed by the battery holder 124. The battery holder 124 may be secured to the PCB 122 such that the battery 126 maintains continuous contact with the PCB 122 and/or is in electrical connection with the components of the PCB 122. The battery 126 may have a particular battery capacity that may affect the life of the battery 126. As will be further discussed below, the distribution of power from the battery 126 to the one or more components of the PCB 122 may be managed to ensure the battery 126 can power the electronics module 120 over the useful life of the inhalation device 100 and/or the medication contained therein.

The electronics module 120 may have a plurality of power states, each with respective power consumption levels. For example, the electronics module 120 may be configured to operate in a system off state, a sleep state, and/or an active state. The system off state may be characterized by very little or no power consumption, while the sleep state may be characterized by greater power consumption than the off state, and the active state may be characterized by greater power consumption than the sleep state. While the electronics module 120 is in the active state, the electronics module may operate in one or more modes, such as a measurement mode, a data storage/data processing mode, an advertising mode, and/or a connected mode. It should be appreciated that the electronics module 120 may operate in multiple modes at one time (e.g., the modes may overlap). For example, as described in more detail below, the electronics modules 120 may operate in the measurement mode and the data storage/data processing mode at discrete times or simultaneously. That is, the electronics module 120 may be perform all of the measurements prior to processing/storing the data, or the electronics module 120 may perform data processing/storage while at the same time also performing additional measurements (e.g., the electronics modules 120 may switch between the measurement mode and the data storage/data processing mode before either is complete).

In the system off state, the electronics module 120 may consume the least amount of power as compared to the other power states (e.g., the sleep state and the active state). In particular, the electronics module 120 may use a minimal amount of power to monitor a certain pin (or pins) on the controller 127 but other components, such as the sensor system 128, the wireless communications circuit 129 (e.g., the Bluetooth radio) and memory may be powered off. The pin on the controller 127 may be in electrical connection with the switch 130 such that actuation of the switch 130 may result in a certain reference signal on the pin. The reference signal may trigger the controller 127 to transition from the system off state.

The system off state may be the initial state of the electronics module 120 after the inhalation device 100 is assembled or manufactured. Thus, the electronics module 120 may be in a system off state prior to the device 100 being delivered to the user and/or prior to the mouthpiece cover 108 being opened for a first time (e.g., before the first use of the inhalation device 100 by the user). In addition, once the mouthpiece cover 108 has been opened for the first time, the electronics module 120 may not return to the system off state thereafter. In some examples, the controller 127 may start its internal clock (e.g., an internal counter) when the electronics module 120 first exits the off state, and any timestamp data generated by the electronics module 120 may be a relative time based on internal clock of the controller. Accordingly, the internal clock may act as a counter that starts when the electronics module 120 exits the off state. Alternatively or additionally, the controller 127 may include an internal system clock that knows the actual time (e.g., 4:05 pm EST on Nov. 18, 2017) and the timestamp data may include the actual time. In such examples, the controller 127 may use power in the off state to run its real-time clock oscillator and to update its system clock.

As noted above, while the electronics module 120 is the active state, the electronics module 120 may operate in one or more modes, such as a measurement mode, a data storage/data processing mode, an advertising mode, and/or a connected mode. In the sleep state, the switch 130 and the controller 127 may continue to receive power from the battery 126, and the controller 127 may continue to run its oscillator and periodically update its system clock (e.g., continue to increment the internal counter that was started when the electronics module 120 first exited the off state). In some examples, the controller 127 may periodically update the system clock every 250 μs.

Further, while in the sleep state, the controller 127 may receive power from the battery to control one or more additional components of the electronics module 120. For example, during the advertising mode, the controller 127 may periodically power on the communications circuit 129 to wirelessly “advertise” to an external device that data is stored on the inhalation device 100 and is available for wireless download. The communications circuit 129 may transmit advertising packets at any interval that is suitable for managing the power consumption of the electronics module 120 when in the sleep state (e.g., as compared to the interval at which packets may be sent during the active state). The advertising interval defines an advertising rate or frequency at which advertising packets are transmitted. For example, advertising packets may be transmitted every 10 seconds when the electronics module 120 is operating in the sleep state. It will be appreciated that the electronics module 120 may spend more time in the sleep state than in any of the other power states. Thus, at a given advertising rate, the electronics module 120 may consume the most power in the sleep state over the life of the inhalation device 100.

In the measurement mode, the controller 127 of the electronics module 120 may power on the sensor system 128. The controller 127 may cause the sensor system 128 to take pressure measurement readings for a predetermined time period (e.g., up to 60 seconds) and/or until the mouthpiece cover 108 is closed or no changes in pressure are detected. The controller 127 may turn off one or more components of the electronics module 120 while the sensor system 128 is capturing pressure measurement readings to further conserve power. The sensor system 128 may sample the pressure at any suitable rate. For example, the sensor system 128 may have a sample rate of 100 Hz and thus a cycle time of 10 milliseconds. The sensor system 128 may generate a measurement complete interrupt after the measurement cycle is complete. The interrupt may “wake” the controller 127 or cause it to turn on one or more components of the electronics module 120. For example, after or while the sensor system 128 is sampling pressure measurements, the controller 127 may process and/or store the pressure measurement data and, if measurements are complete, power off the sensor system 128.

In the data storage/data processing mode, the controller 127 may power on at least a portion of the memory within the electronics module 120. The controller 127 may process the readings from the sensor system 128 to determine airflow metrics and store the airflow metrics in memory. The controller 127 may also compare the readings and/or the airflow metrics to one or more thresholds or ranges to assess how the inhalation device is being used (e.g., whether the pressure readings correspond to no inhalation, a “good” inhalation, to an exhalation, etc.). Depending on the results of the comparison, the controller 127 may drive the indicators to provide feedback to the user of the inhalation device 100. As noted above, the electronics module 120 may operate in the measurement mode and the data storage/data processing mode simultaneously.

In the advertising mode, the controller 127 may power on the communication circuit 129 (e.g., the Bluetooth radio) to advertise to an external device that data is available from the inhalation device 100 and is ready for wireless download. Advertising packets may be transmitted at any interval (respectively, rate) and for any duration that is suitable for managing the power consumption of the electronics module 120 when in the advertising mode. For example, the communications circuit 129 may transmit advertising packets every 100 milliseconds (ms) for 3 minutes. Further, it should be appreciated that the advertising rate may vary based on the particular conditions of the electronics module 120. For example, the advertising rate may be “slow” (e.g., packets are transmitted every 10 seconds) when the electronics module 120 is in the sleep state, whereas the advertising rate may be “fast” (e.g., packets are transmitted every 100 ms) after the measurements and data processing/storage has occurred, such as, for a predetermined amount of time (e.g., 3 minutes) after entering or exiting the active state.

In the connected mode, the communication circuit and memory may be powered on and the electronics module 120 may be “paired” with an external device, such as a smartphone. The controller 127 may retrieve data from the memory and wirelessly transmit the data to the external device. The controller 127 may retrieve and transmit all of the data currently stored in the memory. The controller 127 may also retrieve and transmit a portion of the data currently stored in the memory. For example, the controller 127 may be able to determine which portions have already been transmitted to the external device and then transmit the portion(s) that have not been previously transmitted. Alternatively, the external device may request specific data from the controller, such as any data that has been collected by the electronics module 120 after a particular time or after the last transmission to the external device. The controller 127 may retrieve the specific data, if any, from the memory and transmit the specific data to the external device.

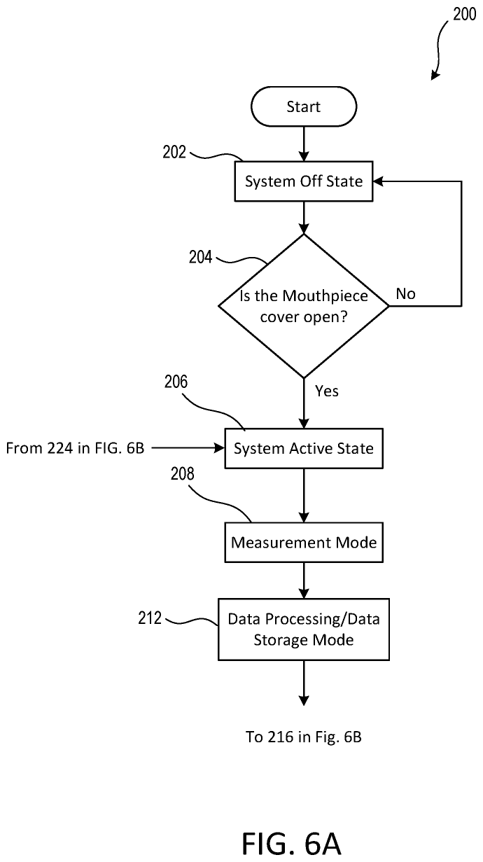

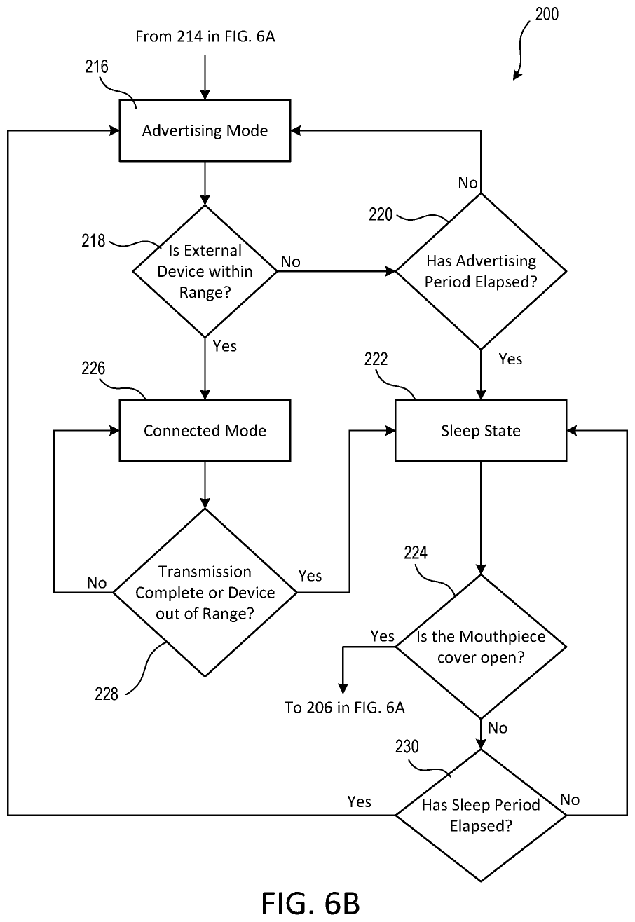

The electronics module 120 may transition between power states or operational modes based on certain conditions or events, such as the position of the mouthpiece cover 108, the actuation of a button, the movement of a lever, the elapse of a predetermined time period, etc. For example, the mouthpiece cover 108 may be closed and the electronics module 120 may be in a system off state or a sleep state. As the mouthpiece cover 108 is moved from the closed position to an open position, the switch 130 may be actuated. The actuation of the switch 130 may cause the electronics module 120 to transition from one state (e.g., the system off state or sleep state) to another state (e.g., the active state). Further, as the actuation of the switch 130 may cause the electronics module 120 to begin operating in one or more operational modes, such as the measurement mode and/or the data storage/data processing mode. For example, FIG. 6A-B illustrate an example flow diagram 200 that illustrates an example process for transitioning between one or more power states and/or operational modes associated with the inhalation device 100.

Further, it should be appreciated that the electronics module 120 may be in the system off state prior to the mouthpiece cover 108 being opened by a user for a first time (e.g., the initial opening of the mouthpiece cover 108 by the user after removing the inhalation device 100 from its packaging). Thereafter, if the mouthpiece cover 108 is returned to the closed state, the electronics module 120 will be in the sleep state (as opposed to the off state). As the user continues to use the inhalation device 100, the electronics module 120 will switch between the sleep state and the active state, based on, for example, one or more events (e.g., an opening/closing of the mouthpiece cover 108, the expiration of a timeout period, the detection of pressure measurements that exceed a threshold (e.g., are indicative of user inhalation), advertising to an external device, etc.).

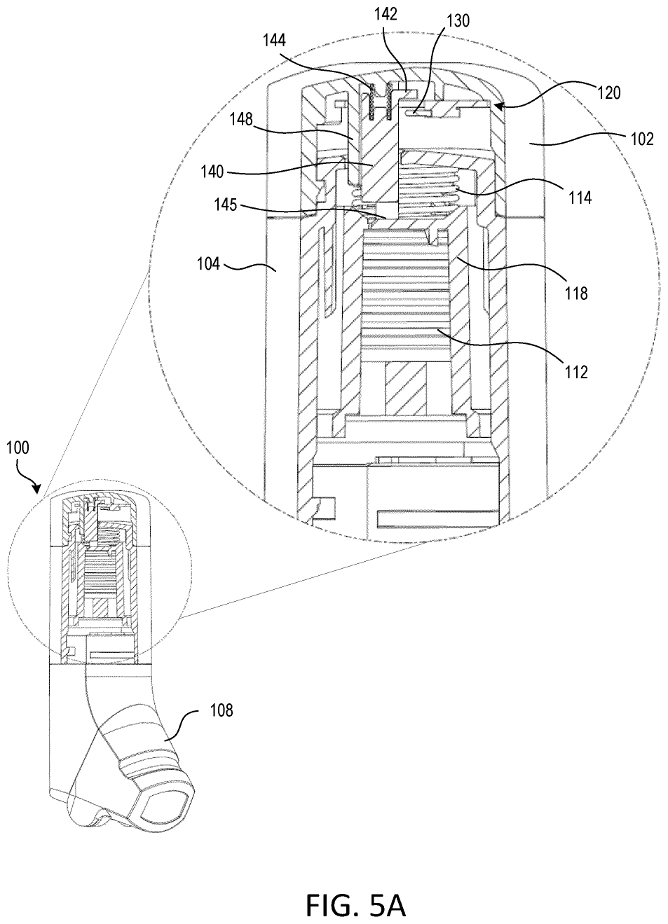

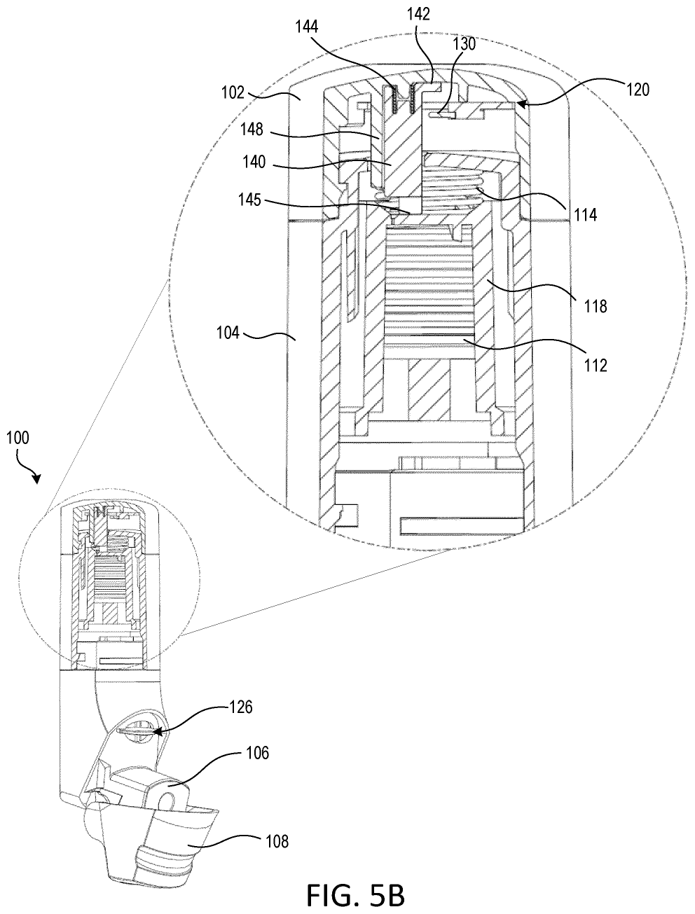

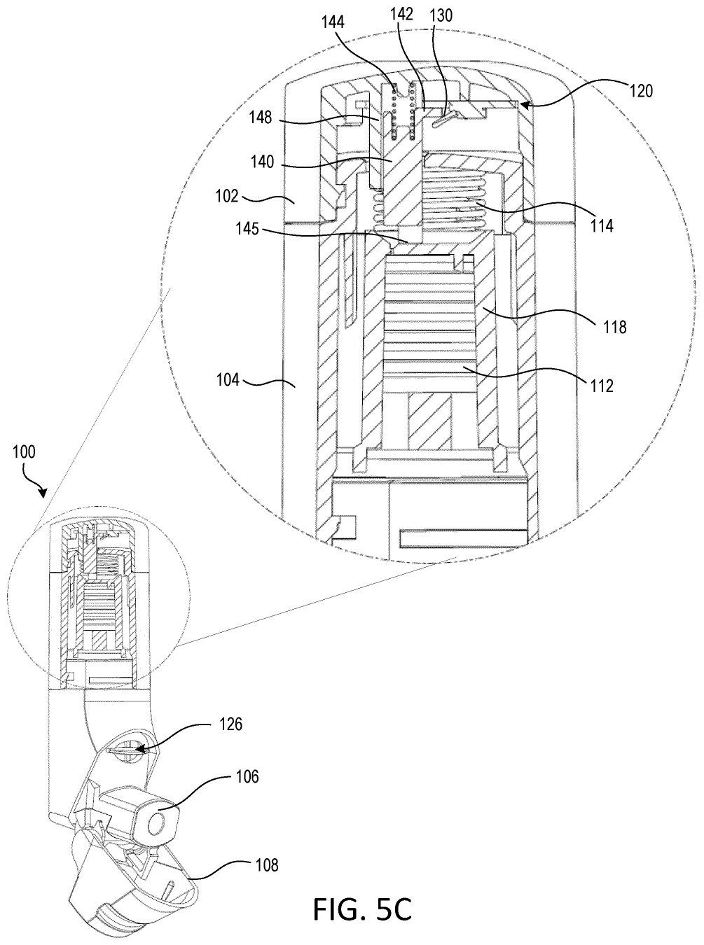

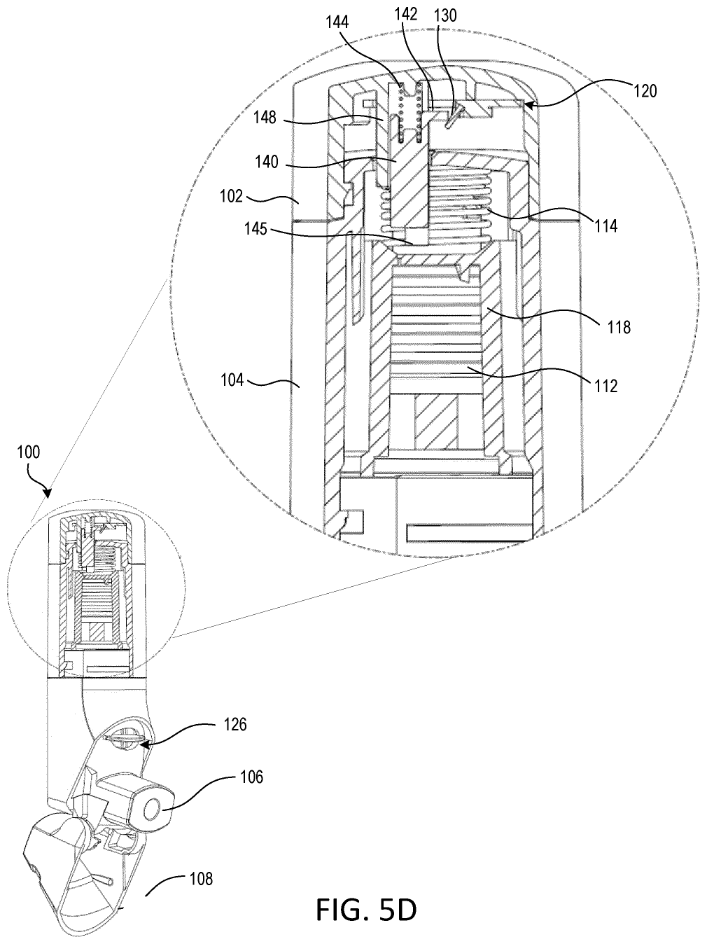

FIG. 5A-5D describe one example of the internal operation of an inhalation device 100. It should be appreciated that other examples of the inhalation device 100 may include a subset of the actions described herein. Referring to FIG. 5A, the distal end 145 of the slider 140 may be configured to abut the yoke 118 that resides within the main housing 104. When the mouthpiece cover 108 is in the closed position, the arm 142 of the slider 140 may not be in contact with the switch 130. Further, the slider spring 144 and the bellows spring 114 may be in a compressed state. As the user begins to open the mouthpiece cover 108 to expose the mouthpiece 106, the yoke 118 may move upward in the main housing 104, for example, due to a mechanical connection between the yoke 118 and the mouthpiece cover 108. The upward movement of the yoke 118 may cause the slider 140 to move upward within the top cap 102, further compressing the slider spring 144 and the bellows spring 114, for example, as shown in FIG. 5B.

As the mouthpiece cover 108 continues to move toward the fully open state, for example as shown in FIG. 5C, the mouthpiece cover 108 may cause the yoke 118 to drop within the main housing 104 (e.g., due to the downward force applied by the bellows spring 114). The movement of the yoke 118 may cause the slider 140 to drop (e.g., due to the downward force applied by the slider spring 144), which may cause the arm 142 of the slider 140 to engage the switch 130 and begin to actuate the switch 130. The downward movement of the slider 140 may be limited by the position of the yoke 118 as the distal end 145 of the slider 140 may rest upon the top of the yoke 118.

As the mouthpiece cover 108 continues to open, as shown in FIG. 5D, the arm 142 of the slider 140 may actuate the switch 130, which may generate a signal causing the electronics module 120 to change states, such as from the off or sleep state to the active state. Thus, the controller 127 of the electronics module 120 may wake and provide power to the sensor system 128 to enable the sensor system 128 to take pressure measurement readings. Moreover, the movement of the yoke 118 caused by the opening of the mouthpiece cover 108 may also cause the yoke 118 to compress the bellows 112 to cause a bolus of medication to be delivered from the medication reservoir 110 to the dosing cup 116, resulting in the medication being made available to the flow channel 119. The medication may be delivered from the dosing cup 116 through the flow channel and out the mouthpiece 106 when a user inhales from the mouthpiece 106.

FIG. 6A-B illustrate an example procedure 200 for transitioning between one or more power states and/or operational modes associated with the inhalation device 100. Although described with reference to the inhalation device 100, any inhalation device may perform the procedure 200. The electronics module 120 of the inhalation device 100 may be in the off state at 202, when the procedure 200 begins. The mouthpiece cover 108 may be in the closed position and the user may not have opened the mouthpiece cover 108 for the first time when the electronics module 120 is in the off state at 202. As noted herein, the off state may be characterized by little or no power consumption by the electronics module 120. At 204, the electronics module 120 may determine whether the mouthpiece cover 108 has been moved into the open position. For example, the opening of the mouthpiece cover 108 may cause the switch 130 to be actuated. If the electronics module 102 determines that the mouthpiece cover 108 has not been moved into the open position, then the electronics module 120 may remain in the off state at 202.

If the electronics module 120 determines that the mouthpiece cover 108 has been moved into the open position at 204, then the electronics module 120 may enter an active state (e.g., a system active state) at 206. The active state may be characterized by greater power consumption than the off state (e.g. and the sleep state). For example, the electronics module 120 may consume more power when in the active state than when in the off state (e.g., and the sleep state). When in the active state, the electronics module 120 may operate in one or more modes, such as a measurement mode, a data storage/data processing mode, an advertising mode, and/or a connected mode. The actuation of the switch 130 may cause the electronics module 120 to transition from the off state to the active state.

While in the active state, and after the mouthpiece cover 108 has been opened, the electronics module 120 may enter a measurement mode at 208. During the measurement mode, the electronics module 120 may power on the sensor system 128 and may cause the sensor system 128 to take pressure measurement readings for a predetermined time period (e.g., up to 60 seconds) and/or until the mouthpiece cover 108 is closed or no changes in pressure are detected.

In examples, the electronics module 120 may remain in the measurement mode until a pressure measurement cycle is complete. The actuation of the switch 130 (e.g., triggered by the opening of the mouthpiece cover 108 may trigger the pressure measurement cycle to start). The pressure measurement cycle may persist for a predetermined period of time and/or until a particular event is detected. For example, the pressure measurement cycle may persist for up to 60 seconds, even if the mouthpiece cover 108 has been closed and the slider 140 has disengaged from the switch 130. Alternatively, the pressure measurement cycle may persist for up to 60 seconds or until the mouthpiece cover 108 has been closed or until no changes in pressure are detected for 10 seconds, whichever comes first. It will be appreciated that the foregoing conditions are merely examples and that any suitable criteria can be used.

At 212, the electronics module 120 may enter a data processing/data storage mode, for example, in response to the reception of data from the sensor system 128 (e.g., the sensor system 128 providing measurements that exceed a threshold, such as pressure measurements that exceed a threshold indication of user inhalation through the inhaler 100). During the data processing/data storage mode, the electronics module 120 may power on at least a portion of the memory within the electronics module 120. The electronics module 120 may process the readings from the sensor system 128 to determine inhalation parameters/metrics and store the inhalation parameters/metrics in memory. The electronics module 120 may also compare the readings and/or the inhalation parameters/metrics to one or more thresholds or ranges to assess how the inhalation device is being used (e.g., whether the pressure readings correspond to no inhalation, a “good” inhalation, to an exhalation, etc.). Depending on the results of the comparison, the electronics module 120 may drive the indicators to provide feedback to the user of the inhalation device 100.

Although not illustrated by the procedure 200, the electronics module 120 may operate in the measurement mode and the data storage/data processing mode simultaneously. For example, the electronics module 120 may switch (e.g., periodically switch) between the measurement mode and the data processing/data storage mode. For example, after or while the electronics module 120 is receiving pressure measurements, the electronics module 120 may process and/or store the pressure measurement data.

The electronics module 120 may remain in the data storage/data processing mode for a predetermined period of time to process and store the pressure measurement readings from the sensor system 128. For example, the electronics module 120 may remain in the data storage/data processing mode for up to 60 ms. The electronics module 120 may, for example, use up to 50 ms to process and compute airflow metrics from the pressure measurement readings and up to 10 ms to store the pressure measurements and/or airflow metrics in the memory. Alternatively, the electronics module 120 may remain in the data storage/data processing mode for whatever duration it takes for the controller 127 to process and store the pressure measurement readings and/or air flow metrics.

The electronics module 120 may enter the advertising mode at 216. For example, the electronics module 120 may enter the advertising mode after the predetermined period of time for data processing and data storage has elapsed, when the mouthpiece cover 108 is closed (e.g., returned to a closed position), and/or after the controller 127 has determined that such processing and storing are complete. In the advertising mode, the electronics module 120 may power on the communication circuit 129 (e.g., the Bluetooth radio) to advertise to an external device that data is available from the inhalation device 100 and is ready for wireless download. Advertising data (e.g., advertising packets) may be transmitted at any interval and for any duration that is suitable for managing the power consumption of the electronics module 120 when in the advertising mode. For example, the communications circuit 129 may transmit advertising data (e.g., over an advertising channel) at an advertising rate of once every 100 milliseconds (ms) for an advertising period. The advertising period may be a period of time that the electronics module 120 continues to advertise to an external device before changing power states. The advertising period may be three minutes. An advertising period may be partitioned into shorter advertising periods. Each of the partitioned advertising periods may be the same length and may sum to the full advertising period. The partitioned advertising periods may be consecutive such that one partitioned advertising period begins when another partitioned advertising period ends. Additionally or alternatively, the partitioned advertising periods may be separated in time such that there is a time delay between successive partitioned advertising periods.

Further, it should be appreciated that the advertising rate may vary based on the particular conditions of the electronics module 120. Additionally or alternatively, the advertising rate may vary based on a data transmission status. For example, the electronics module 120 may adjust the advertising rate based on a failed data transfer. The advertising rate may be “slow” (e.g., packets are transmitted every 10 seconds) when the electronics module 120 is in the sleep state and without the mouthpiece cover 108 moving to the open position (e.g., when transitioning from 230 to 216), whereas the advertising rate may be “fast” (e.g., data is transmitted every 100 ms) after the measurements and data processing/storage has occurred (e.g., when transitioning from 212 to 216). For example, the advertising rate may be “fast” when the processed data is ready to be sent to an external device. Using the slower advertising rate in the sleep state (e.g., when the inhaler is not in use and/or the mouthpiece cover 108 is closed) may save resources and increase battery life of the electronics module and the external device while maintaining connection and pressure measurement capabilities. Additionally or alternatively, partitioned advertising periods may be associated with the same or different advertising rates. For example, the electronics module 120 may adjust the advertising rate between successive partitioned advertising periods (e.g., based on an energy level of the battery 130, a failed data transfer, and/or a state of the inhalation device).

At 218, the electronics module 120 may determine if an external device is within range. If the external device does not come within a particular range of the electronics module 120 during the advertising mode, the electronics module 120 may determine whether an advertising period (e.g., 3 minutes) has elapsed at 220. If the advertising period has not elapsed, then the electronics module 120 may continue to transmit advertising data (e.g., over an advertising channel) to the external device at 216. However, if the advertising period has elapsed, then the electronics module 120 may move to a sleep state at 222. The sleep state may be characterized by greater power consumption than the off state, but less power consumption than the on state. For example, the electronics module 120 may consume more power in the sleep state than in the off state and the electronics module 120 may consume less power in the sleep state than in the on state.

The electronics module 120 may remain in the sleep state for a predetermined amount of time and/or until the electronics module determines that the mouthpiece cover 108 has been moved from the closed to the open position. For example, the electronics module 120 may periodically switch between the sleep state and the advertising mode (e.g., the slow advertising mode or the fast advertising mode) of the active state. For example, at 224, the electronics module 120 may determine whether the mouthpiece cover 108 has been moved from the closed to the open position. If the mouthpiece cover 108 has been moved into the open position, then the electronics module 120 may enter the active state at 206. For example, the opening of the mouthpiece cover 108 may cause the switch 130 to be actuated. The actuation of the switch 130 may cause the electronics module 120 to transition from the sleep state to the active state. In addition, the actuation of the switch 130 may initiate a timer (e.g., an advertising period timer). The timer may be configured to expire a set time period (e.g., 180 seconds) after actuation of the switch 130.

If the electronics module 120 determines that the mouthpiece cover 108 remains in the closed position, then the electronics module 120 may determine whether a sleep period (e.g., 10 seconds) has elapsed at 230. If the sleep period has not elapsed at 230, then the electronics module 120 may stay in the sleep state and return to 222. However, if the sleep period has elapsed at 230, then the electronics module 120 may return to the advertising mode of the active state at 216. When the electronics module 120 transitions from 230 to 216, the electronics module 120 may advertise data at a different (e.g., faster or slower rate) as compared to when the electronics module 120 transitions from 212 to 216 (e.g., such as once every 100 ms as opposed to once every 10 seconds). As such, the electronics module 120 may use less battery power during such advertising modes. In addition, the electronics module 120 may use more available battery power to perform pressure measurements and generate dose records for subsequent dosing events. Further, the electronics module 120 may periodically switch between the active state and the sleep state based on the advertising period and the sleep period (e.g., and while the mouthpiece cover 108 is in the closed position).

Returning to 218, if the external device (e.g., smartphone or tablet) comes within a particular range of the electronics module 120 during the advertising mode, the electronics module 120 may “pair” with the external device and enter a connected mode at 226. The connected mode may also be referred to as a connected state herein. In the connected mode, the electronics module 120 may power on the communication circuit and memory. The electronics module 120 may retrieve data from the memory and cause the communication circuit to wirelessly transmit the data to the external device. At 228, the electronics module 120 may determine whether the transmission of the data is complete or the external device is out of communication range. When the transmission is not complete and/or the external device is within the communication range, the electronics module 120 may remain in the connected mode. When the transmission is complete and/or if the external device is out of the communication range, the electronics module 120 may transition to the sleep state at 222.

During the connected mode, the electronics module 120 may retrieve and transmit data (e.g., all of the data) currently stored in the memory. For example, the electronics module 120 may retrieve and transmit a portion of the data currently stored in the memory. For example, the electronics module 120 may determine which portions of data have already been transmitted to the external device (e.g., based on the internal counter) and then transmit the one or more portion(s) of data that have not been previously transmitted. Alternatively or additionally, the external device may request specific data from the electronics module 120, such as any data that has been collected by the electronics module 120 after a particular time or since a last transmission to the external device. The electronics module 120 may retrieve the specific data, if any, from the memory and transmit the specific data to the external device.

Further, when connected (e.g., in the connected mode) with the external device, the electronics module 120 may be configured to transmit Bluetooth special interest group (SIG) characteristics for managing access to records stored in the electronics module 120. The Bluetooth SIG characteristics may include one or more of a manufacturer name of the inhalation device 100, a serial number of the inhalation device 100, a hardware revision number of the inhalation device 100, and/or a software revision number of the inhalation device 100. Alternatively or additionally, in some examples, the electronics module 120 may be configured to include any combination of the manufacturer name of the inhalation device 100, the serial number of the inhalation device 100, the hardware revision number of the inhalation device 100, and/or the software revision number of the inhalation device 100 within one or more advertising packets (e.g., that are sent prior to entering the connected mode). When connected with the external device, the electronics module 120 may retrieve data from memory and transmit the data to the external device.

The inhalation device 100 (e.g., using the communication circuit of the electronics module 120) may transmit data that includes or is associated with an inhalation event, an inhalation parameter, a pressure measurement, a mouthpiece cover 108 event, an error event, an operating characteristic of the inhalation device (e.g., remaining battery life), a dosing event, a dose record, and/or associated timestamps (e.g., based on the internal counter) to the external device when in the connected mode. For example, the dose record, the signals generated by the switch 130, the pressure measurement readings taken by the sensory system 128, and/or the airflow metrics computed by the controller 127 of the electronics modules 120 may be timestamped and stored in memory. The foregoing data may be indicative of various usage parameters associated with the inhalation device 100. For example, as movement of the slider 140 causes the switch 130 to transition between “on” and “off”, the controller 127 of the electronics module 120 may use the signals from the switch 130 to record and timestamp each transition. Further, as the transition of the switch 130 between “on” and “off” may correlate to the position of the mouthpiece cover 108 (e.g., open or closed), the electronics module 120 may detect and track the position of the mouthpiece cover 108 over time. It will be appreciated that the electronics module 120 may sense and track the status of the mouthpiece cover 108 without interfering with the delivery of medication through the flow pathway 119 of the inhalation device 100.

The pressure measurement readings and/or the computed airflow metrics may be indicative of the quality or strength of inhalation from the inhalation device 100. For example, when compared to a particular threshold or range of values, the readings and/or metrics may be used to categorize the inhalation as a certain type of event, such as a good inhalation event, a low inhalation event, a no inhalation event, or an excessive inhalation event.

The no inhalation event may be associated with pressure measurement readings and/or airflow metrics below a particular threshold, for example, such as an airflow rate less than 30 Lpm. The no inhalation event may occur when a user does not inhale from the mouthpiece 106 after opening the mouthpiece cover 108 and during the measurement cycle. The no inhalation event may also occur when the user's inspiratory effort is insufficient to ensure proper delivery of the medication via the flow pathway 119, such as when the inspiratory effort generates insufficient airflow to activate the deagglomerator 121 and, thus, aerosolize the medication in the dosing cup 116.

The low inhalation event may be associated with pressure measurement readings and/or airflow metrics within a particular range, for example, such as an airflow rate between 30 Lpm and 45 Lpm. The low inhalation event may occur when the user inhales from the mouthpiece 106 after opening the mouthpiece cover 108 and the user's inspiratory effort causes at least a partial dose of the medication to be delivered via the flow pathway 119. That is, the inhalation may be sufficient to activate the deagglomerator 121 such that at least a portion of the medication is aerosolized from the dosing cup 116.

The good inhalation event may be associated with pressure measurement readings and/or airflow metrics above the low inhalation event, for example, such as an airflow rate between 45 Lpm and 200 Lpm. The good inhalation event may occur when the user inhales from the mouthpiece 106 after opening the mouthpiece cover 108 and the user's inspiratory effort is sufficient to ensure proper delivery of the medication via the flow pathway 119, such as when the inspiratory effort generates sufficient airflow to activate the deagglomerator 121 and aerosolize a full dose of medication in the dosing cup 116.

The excessive inhalation event may be associated with pressure measurement readings and/or airflow metrics above the good inhalation event, for example, such as an airflow rate above 200 Lpm. The excessive inhalation event may occur when the user's inspiratory effort exceeds the normal operational parameters of the inhalation device 100. The excessive inhalation event may also occur if the device 100 is not properly positioned or held during use, even if the user's inspiratory effort is within a normal range. For example, the computed airflow rate may exceed 200 Lpm if the air vent 126 is blocked or obstructed (e.g., by a finger or thumb) while the user is inhaling from the mouthpiece 106.

It will be appreciated that any suitable thresholds or ranges may be used to categorize a particular event. It will further be appreciated that some or all of the events may be used. For example, the no inhalation event may be associated with an airflow rate below 45 Lpm and the good inhalation event may be associated with an airflow rate between 45 Lpm and 200 Lpm. As such, the low inhalation event may not be used in some cases.

The pressure measurement readings and/or the computed airflow metrics may also be indicative of the direction of flow through the flow pathway 119 of the inhalation device 100. For example, if the pressure measurement readings reflect a negative change in pressure, the readings may be indicative of air flowing out of the mouthpiece 106 via the flow pathway 119. If the pressure measurement readings reflect a positive change in pressure, the readings may be indicative of air flowing into the mouthpiece 106 via the flow pathway 119. Accordingly, the pressure measurement readings and/or airflow metrics may be used to determine whether a user is exhaling into the mouthpiece 106, which may signal that the user is not using the device 100 properly.

By timestamping and storing the signals generated by the switch 130, the pressure measurement readings taken by the sensory system 128, and/or the airflow metrics computed by the controller 127 of the electronics module 120, the data collected and stored by the electronics module 120 may be used to determine whether the usage parameters are suitable or appropriate over a given period of time. As such, the data may be indicative of other events, such as an overuse event, an underuse event, or an optimal use event.

For example, the user of the inhalation device 100 may be prescribed by his or her doctor to take two doses of medication via the inhalation device 100 each day. In addition, the medication contained in the inhalation device 100 may also be approved (for safety and regulatory purposes) to be taken no more eight times each day. The overuse event may occur if the electronics module 120 records more than two good inhalations in a twenty-four hour period (i.e., the actual dosing is exceeding the prescribed number of doses) and/or if the electronics module 120 records more than eight good inhalations in a twenty-four hour period (i.e., the actual dosing is exceeding the regulatory approved number of doses). The underuse event may occur if the electronics module 120 records less than two good inhalations in a twenty-four hour period (i.e., the actual dosing is below the prescribed number of doses). The optimal use event may occur if the electronics module 120 records two good inhalations in a twenty-four hour period (i.e., the actual dosing is below the prescribed number of doses). It will be appreciated that optimal use events may be indicative of a user who is adherent. It will further be appreciated that the prescribed dosing schedule and/or the maximum approved dosing schedule may depend on the type of medication contained in the inhalation device 100. In addition, the events may be defined using any suitable number of doses over any suitable period of time, such as two doses per day, fourteen doses per week, 60 doses per month, etc.

The data collected and stored by the electronics module 120 may also be used to estimate the number doses that have been delivered from the inhalation device 100 and/or estimate the number of doses that remain in the medication reservoir 110. For example, each time the switch 130 is activated via the opening of the mouthpiece cover 108, the signal generated by the switch 130 may be counted as a dosing event. The dosing event may include actuation of the switch 130 (e.g., via opening of the mouthpiece cover 108) and/or receipt of measurements (e.g., measurements that exceed a threshold that, for example, is indicative of a user's inhalation through the inhaler). Thus, the inhalation device 100 may be deemed to have delivered 60 doses when the mouthpiece cover 108 is opened 60 times. The inhalation device 100 may be configured to store enough medication in the medication reservoir 110 to deliver a predefined total number of doses, such as a total of 200 doses. As such, the inhalation device 100 may also be deemed to have 140 doses remaining after the mouthpiece cover 108 is opened 60 times.

As noted above, medication will not be delivered from the medication reservoir 110 upon the user opening the mouthpiece cover 108 if a previous dose of medication was not properly aerosolized by the deagglomerator 121 and/or transferred from the dosing cup 116. Thus, it will be appreciated that counting the number of doses based on the opening of the mouthpiece cover 108 may not accurately reflect the actual number of doses delivered by the device 100 if, for example, a user opens and closes the mouthpiece cover 108 without inhaling from the mouthpiece 106. Accordingly, other data in the electronics module 120 may be used and/or combined with the signals from the switch 130 to determine the number of doses delivered and/or remaining in the deice 100. For example, a dose may be counted as delivered each time a computed airflow metric is above a threshold or within a particular range, such as when a good inhalation event has been recorded. By calculating and tracking the number of doses delivered and/or remaining, the electronics module 120 may be configured to identify a refill event, which may be indicative of a time when a user should consider obtaining a new inhalation device 100.

The data collected and stored by the electronics module 120 may also be used to determine various error conditions associated with the operation of the module 120. For example, when processing the data the electronics module 120 may generate a bad data flag, a data corrupt flag, a timestamp error flag, and/or the like. The electronics module 120 may generate the bad data flag when the controller 127 of the electronics module 120 determines that one or more signals received from the sensor system 128 are outside a predetermined range, which may indicate a malfunction in the sensor system 128. The electronics module 120 may generate the data corrupt flag when the controller's cyclic redundancy check (CRC) of data does not match what is stored in memory, which may indicate a malfunction of the memory and/or that the data in the memory has been corrupted. The electronics module 120 may generate a timestamp error flag when the controller 127 loses its electrical connection with the battery 126, causing the controller's system clock to reset. If the controller's system clock is reset, the controller 127 may restart its clock from the last stored counter value.

The electronics module 120 (e.g., and/or a mobile application residing on an external device) may also analyze the recorded events over a period of time to identity multiple error events, which may include a pattern of use indicative of a user who is not familiar with the proper operation of the inhalation device 100 and thus a user who may require further training. For example, the electronics module 120 may look at the number of good inhalation events over a predetermined period of time and/or over a predetermined number of openings of the mouthpiece cover 108. A multiple error event may occur when a user has had only two good inhalation events over the past week, or has had six or less good inhalations over the last twelve openings of the mouthpiece cover 108. It will be appreciated that the foregoing conditions are merely exemplary and that any suitable pattern of use may be used to define a multiple error event.

The data collected and stored by the electronics module 120 may also be used to assess the amount of power remaining in the battery 126. For example, the controller 127 may determine whether there is a low battery event or condition, such as whether the battery has less than a predetermined amount of charge remaining (e.g., below 10%).

It will be appreciated that electronics module 120 may process and analyze the data stored in memory (e.g., the signals generated by the switch 130, the pressure measurement readings taken by the sensory system 128 and/or the airflow metrics computed by the controller 127 of the PCB 122) to determine the usage parameters associated with the inhalation device 100. For example, the electronics module 120 may process the data to identify no inhalation events, low inhalations events, good inhalation events, excessive inhalation events and/or exhalation events. The electronics module 120 may also process the data to identify underuse events, overuse events and optimal use events. The electronics module 120 may further process the data to estimate the number of doses delivered and/or remaining and to identify error conditions, such as those associated with a timestamp error flag. The electronics module 120 may inform the user of some or all of the foregoing usage parameters of the inhalation device 100 using the indicators, such as one or more LEDs. As an example, the electronics module 120 may illuminate an LED to indicate a good inhalation event or change the color of an LED to indicate a low inhalation event or a no inhalation event. The usage parameters may be indicated to the user via any combination of light sequences and/or light color schemes.

It will further be appreciated that the data stored in the memory of the electronics module 120 (e.g., the dose records, the signals generated by the switch 130, the pressure measurement readings taken by the sensory system 128 and/or the airflow metrics computed by the controller 127 of the electronics module 120) may also be transmitted to an external device, which may process and analyze the data to determine the usage parameters associated with the inhalation device 100. Further, a mobile application residing on the mobile device may generate feedback for the user based on data received from the electronics module 120. For example, the mobile application may generate daily, weekly, or monthly report, provide confirmation of error events or notifications, provide instructive feedback to the user, and/or the like. A dose record may include any combination of data that is associated with a dosing event of the user (e.g., associated with each user interaction and use of the inhalation device 100). For example, the dose record may include any combination of a dose count, an inhalation start time, an inhalation duration, an inhalation peak, a time-to-peak, an inhalation volume, a switch actuation start time, a switch actuation close time, and/or any combination of status flags.

In some examples, the inhalation device 100 may be configured to communicate a dose count (e.g., the total dose count) and/or one or more dose records to the external device in the advertising data that is communicated by the inhalation device 100. For example, the advertising data may include indications of the dose count of the inhalation device 100 and/or one or more dose records. As such, before entering a connected state with the external device (e.g., which may increase battery usage), the external device may ensure that there is new information to be communicated by the inhalation device 100. This may ensure that the inhalation device 100 does not unnecessarily enter a connected state with the external device when no new data exists (e.g., all existing data in the memory of the inhalation device has already been transmitted to the external device), which in turn may save battery power of the inhalation device 100.

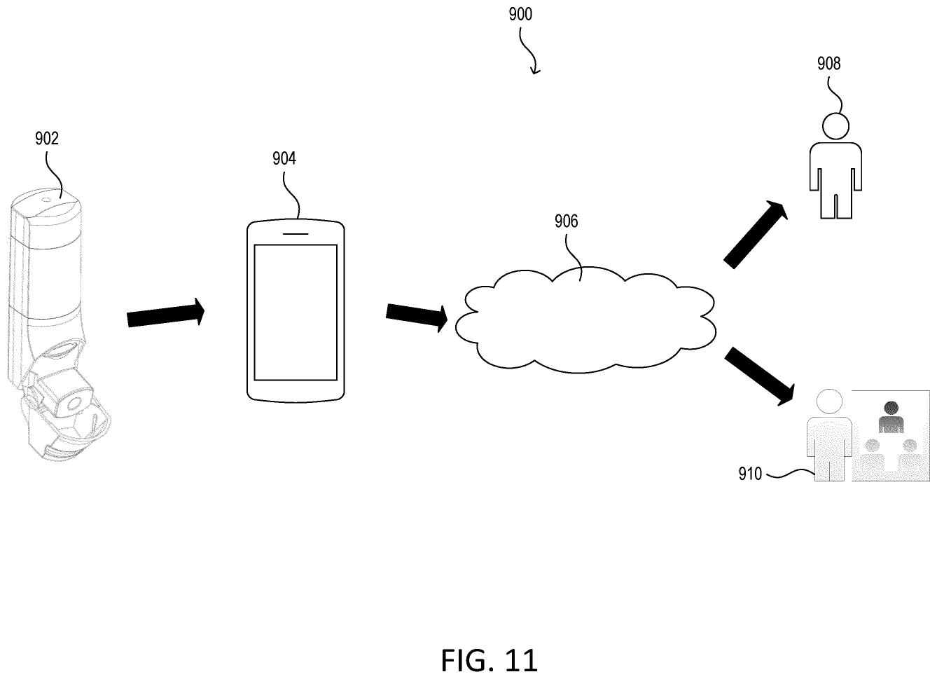

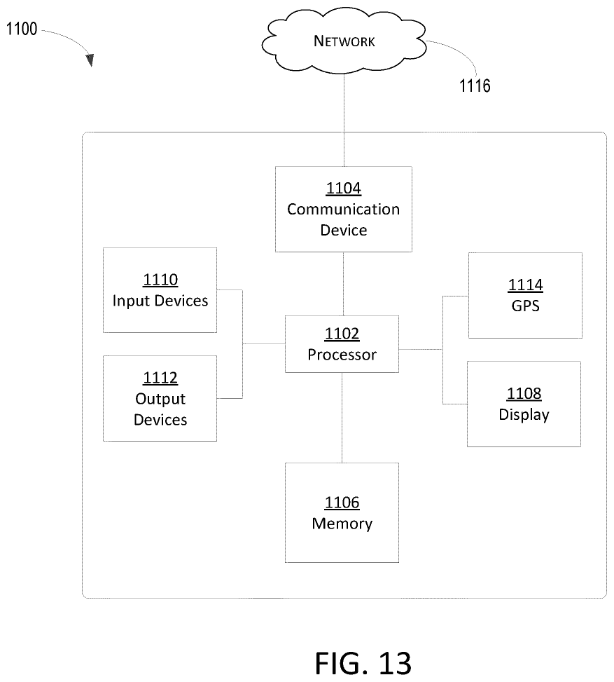

FIG. 7 illustrates an example procedure 300 for an external device (e.g., such as the external device 904 shown in FIG. 11 and/or the external device 1100 shown in FIG. 13) to determine whether to connect with an inhalation device (e.g., such as the inhalation device 100 shown in FIGS. 1-4 and 5A-5D). The external device may use the procedure 300 to determine whether to connect to the inhalation device, for example, to save resources (e.g., battery life) of the inhalation device and/or external device by avoiding unnecessary advertisements and/or connections between the inhalation device and the external device. The inhalation device may comprise an electronics module (e.g., such as the electronics module 120 shown in FIGS. 2-4 and 5A-5D). The electronics module may be in the sleep state or the active state at 302, when the procedure 300 begins. The external device may be a mobile device, a personal computer, a smart hub, etc. A controller (e.g., processor) of the external device may perform the procedure 300 periodically and/or in response to the reception of an advertising packet.

At 302, the external device may receive advertising data from a communication circuit, for example, of an inhalation device (e.g., such as the communication circuit 129 of the inhalation device 100 shown in FIG. 3). The advertising data may indicate whether the inhalation device has new recorded dose records stored thereon (e.g., dose records that have yet to be communicated out from the inhalation device). For example, the advertising data may include one or more dose record indicators. The dose record indicator may indicate a dose record that is stored in the memory of the electronics module. For example, the dose record indicator may indicate to the external device that the one or more dose records are available from the inhalation device and is ready for wireless download. For instance, the dose record indicator may indicate the most recently stored dose record(s) in memory. The dose record indicator may indicate a dose count of the inhalation device, a total number of dose records stored in the memory of the inhalation device, and/or an indication of a present dose count of a dosing record of the inhalation device. In some examples, the dose record indicator may include one or more dose flags relating to the inhalation device. For example, the dose record indicator may include an indication of the number of doses that were recorded by the inhalation device since the last time it transferred data to an external device, a present count of the internal counter of the electronics module, the state of the mouthpiece cover (e.g., opened or closed), etc.

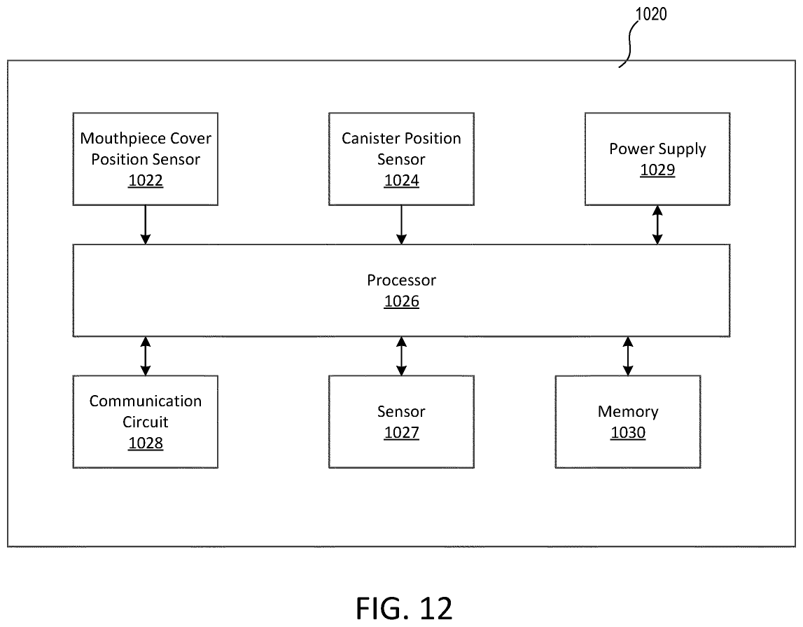

For instance, the electronics module of the inhalation device may generate a dose record in response to a dosing event (e.g., the detection of a dosing event). The dosing event may include any interaction the user has with their inhalation device when inhaling. For example, the dosing event may include actuation of a switch of the inhalation device (e.g., such as switch 130 shown in FIGS. 1 and 5A-5D) and/or receipt of measurements by a sensor of the inhalation device (e.g., such as sensor system 128 shown in FIG. 3 and/or sensor 1027 shown in FIG. 12). For example, the processor of the electronics module (e.g., such as processor 1026 shown in FIG. 12) may receive pressure measurements from the sensor that are indicative of an inhalation. The electronics module may determine that a dosing event occurred when the pressure measurements are greater than a threshold pressure. The threshold pressure may be indicative of a user's inhalation via the mouthpiece of the inhaler.

The electronics module may be configured to store the generated dose record in the memory of the inhalation device. The dose record may be stored with other dose records (e.g., dose records that have and that have not been previously transmitted by the communication circuit). The dose record may include any combination of an inhalation start time, an inhalation duration, an inhalation peak, a time-to-peak, an inhalation volume, a switch actuation start time, a switch actuation close time, a dose status flag, and/or associated time stamps.

The electronics module may transmit advertising data that comprises the dose record indicator. This can enable the external device to obtain information about the dose record with minimal delay and with minimal power consumption by both devices. The information can be obtained without polling of the electronics module by the external device, and without the need for the two devices to enter a connected state, for example. Providing the dose record indicator in the advertising data may assist the external device to rapidly determine whether the dose record comprises new data, not previously seen by the external device. It can achieve this without requiring the external device and the communication circuit to enter a connected state (which would result in additional power consumption, by both devices). The external device need only interrogate the electronics module (e.g., by sending a scan request or entering a connected state with the communication circuit) if it determines that it requires further information, such as the dose record itself.

For example, the processor may cause the communication circuit to transmit advertising data that comprises the dose record indicator. The advertising data may include data in addition to the dose record indicator. For instance, the advertising data may include any combination of the manufacturer name of the inhalation device, the serial number of the inhalation device 100, the hardware revision number of the inhalation device, and/or the software revision number of the inhalation device within one or more advertising packets. In some examples, the advertising data may include one or more Universally Unique ID (UUID) specific to the inhalation device. The advertising data may include the medication type of the inhalation device, the dose strength of the inhalation device, the number of doses (e.g., the size) of the inhalation device, and/or a serial number of the inhalation device.

The advertising data (e.g., advertising packets) may be transmitted at any interval and for any duration that is suitable for managing the power consumption of the electronics module when in the advertising mode. In general, the higher the advertising rate, the greater the associated power consumption by the communications circuit. Conversely, the lower the advertising rate, the lower the power consumption by the communications circuit. This leads to a tradeoff between latency and power consumption (since a fast advertising rate generally results in reduced latency of communication). The competing requirements may be balanced in a variety of ways. For example, the communications circuit may transmit advertising data at a first advertising rate or a second advertising rate based on one or more conditions of the electronics module and/or a data transmission status. The one or more conditions of the electronics module may include a battery energy level. The data transmission status may include a data transmission failed status. The first advertising rate may be once every 100 ms and the second advertising rate may be once every 10 seconds. It should be appreciated that the first and second advertising rates are not limited to the specific rates specified herein.

Further, in some examples, the external device may send a scan request to the inhalation device, for example, in response to the reception of the advertising data and/or in response to the determination that the dose record has not previously been transmitted by the electronics module (e.g., to the external device). The inhalation device may receive the scan request, and in response, send a scan response message. The external device may receive the scan response message from the inhalation device, and the scan response message may include a plurality of dose record indicators, such as the last two dose records, generated by the inhalation device (e.g., in addition to or in lieu of the inclusion of the dose record indicators in the advertising data). Each dose record indicator in the scan response message may include any combination of an inhalation start time, an inhalation duration, an inhalation peak, a time-to-peak, an inhalation volume, a switch actuation start time, a switch actuation close time and/or a dose status flag for each of one or more (e.g., a plurality) of dose records. In this way, the external device can obtain additional information about dose records of the inhaler without necessarily entering a connected state with the communication circuit. In general, the use of the scan request and scan response message is associated with lower power consumption requirements, compared with entering a connected state. This kind of interaction is generally also quicker than negotiating a connected state. Consequently, the use of the scan request and scan response message can help to reduce power consumption and latency.

At 304, the external device may determine, based on the advertising data, whether the inhalation device comprises one or more new dose records stored within memory. For example, the external device may determine that there are new records when the dose count associated with the inhalation device has changed. For example, the external device may compare the dose count received in the advertising data against a most recent dose count stored in memory of the external device. The external device may determine that the dose count has changed when one or more of the dose records have not previously been received by the external device. The external device may determine that the inhalation device has a new dose record based on any of the data included within the dose record indicator, such as, an indication (e.g., explicit indication) that new doses exist, the counter value of an indicated dose record, etc.

If the external device determines, at 304, that the inhalation device has at least one dose record that is not stored on the external device (e.g., the dose count associated with the inhalation device has changed), the external device may connect to the inhalation device at 306 (e.g., the communication circuit of the electronics module). That is, the external device may determine whether to connect, at 306, to the inhalation device based on information (e.g., the dose record indicator) included in the advertising data received from the inhalation device. For example, the external device may enter a connected state with the inhalation device at 306. The external device may be “paired” with the inhalation device in the connected state. In examples that utilize the scan request and response messages, the external device may determine to enter the connected state with the inhalation device in response to a determination that at least one dose record included in the scan response message has not been previously received by the external device (e.g., transmitted by the electronics module). The external device may receive the one or more dose records from the inhalation device in the connected state. The procedure 300 may end when the external device is in the connected state with the inhalation device. The external device may repeat the procedure 300 in respond to receiving advertising data from the inhalation device. According to such an example, power consumption (by both the inhaler and the external device) may be reduced by only entering the connected state when one or more new dose records, stored by the inhaler, have not yet been received by the external device.

If, however, the external device determines that the inhalation device does not have at least one dose record that is not stored on the external device at 304 (e.g., that the inhalation device has no new dose records), the external device may exit the procedure 300. For example, the external device may exit the procedure, without entering a connected state with the inhalation device, when the inhalation device does not include dose records not already stored on the external device. As such, the procedure 300 allows for the external device to avoid entering a connected state with the inhalation device when the inhalation device does not include any new dose records, which for example, may prevent unnecessary communication between the devices and/or prolong the battery life of the inhalation device. By using the advertising data to communicate the most recent dose records stored in memory, the inhalation device can ensure that it does not unnecessarily enter a connected state with an external device when no new data exists (e.g., all existing data in the memory of the inhalation device has already been transmitted to the external device).

As described in more detail herein, the inhalation device (e.g., the inhalation device 100) may be configured to generate a dose record in response to a dosing event where, for example, the dosing event may include the actuation of a switch of the electronics module and/or be based on measurements received from the sensor of the electronics module of the inhalation device. The inhalation device may store the dose record in memory of the electronics module and attempt to transmit the dose record to an external device. The inhalation device may leverage multiple (e.g., two) different advertising rates when attempting to establish a wireless connection with an external device. For example, the inhalation device may be configured to transmit advertising data at a fast advertising rate for a predetermined period of time after the detection of a dosing event and/or generation of a dose record, and fall back to a slow advertising rate thereafter to, for example, find a balance between fast data transmission and a conservation of battery power. The initial, fast advertising rate facilitates communication with an external device as quickly as possible, if such an external device is within range and ready to detect the advertising transmissions. Limiting the use of the fast advertising rate to the predetermined period helps to conserve battery power, since the use of the fast advertising rate is relatively energy-intensive. The subsequent, slower advertising rate helps to conserve battery power, it is less energy-intensive than the fast advertising rate. At the same time, because the inhalation device continues to transmit advertising data (albeit less frequently), communication with an external device remains possible—for example, when an external device subsequently comes into range.

However, even if the inhalation device successfully connects with the external device, data transmission may not always be successful. For example, the transmission of the dosing record may fail and/or the connection may be lost. In some examples, the inhalation device may be configured to transmit advertising data at a fast advertising rate in response to a failed data transmission, for example, in an effort to expedite the reconnection to the external device after a failed data transfer. But the inhalation device may fall back to a slow advertising rate after some time period (e.g., after the expiration of multiple advertising periods). Although the use of the fast advertising rate may result in an increase of the battery power usage of the inhalation device, the inhalation device may be configured to attempt to reconnect with the external device by sending advertising data at a fast advertising rate in response to a failed data transmission since, for example, the inhalation device and the external device may still be in communication range of one another.

For instance, the inhalation device may be configured to set a retry value and an advertising period timer in response to the dosing event. The advertising period timer may be set to a time period (e.g., 60 seconds) that the inhalation device will transmit advertising data at a fast advertising rate (e.g., once every 10 ms). The inhalation device may compare the retry value to a retry threshold (e.g., three, if the retry value is initially set to zero and counts upwards, or zero, if the retry value is initially set to three and counts down) at the expiration of the advertising period timer and/or a failed data transmission. The retry threshold may represent the number of times the inhalation device is configured to restart the advertising period timer before falling back to a slow advertising rate (e.g., once every 10 seconds). For instance, the inhalation device may be configured to adjust (e.g., decrease) the retry value in response to a failed data transmission or in response to the expiration of the advertising period timer. The inhalation device may be configured to reset the advertising period timer based on a determination that the retry value is, for example, greater than a retry threshold. As such, the inhalation device may be configured to cause the communication circuit to transmit advertising data at the fast advertising rate during the pendency of the advertising period timer. However, the inhalation device may cause the communication circuit to transmit advertising data at the slow advertising rate upon the expiration of the advertising period timer and a determination that the retry value is, for example, less than or equal to the retry threshold. Accordingly, the inhalation device may be configured to fall back to the slow advertising rate in response to a determination that the inhalation device has attempted to transmit advertising data at the fast advertising rate for a number of advertising periods (e.g., three) without a successful data transmission (e.g., of a dosing event), regardless of whether there was an successful connection established between the inhalation device and the external device. The number of advertising periods is determined by the difference between (i) the retry value immediately after it is set and (ii) the retry threshold. As mentioned already above, it should be understood that the retry value may count upwards (that is, it may be adjusted by incrementing) or it may count downwards (that is, it may be adjusted by decrementing). The threshold and comparison tests can be selected according to the chosen mode of adjustment.

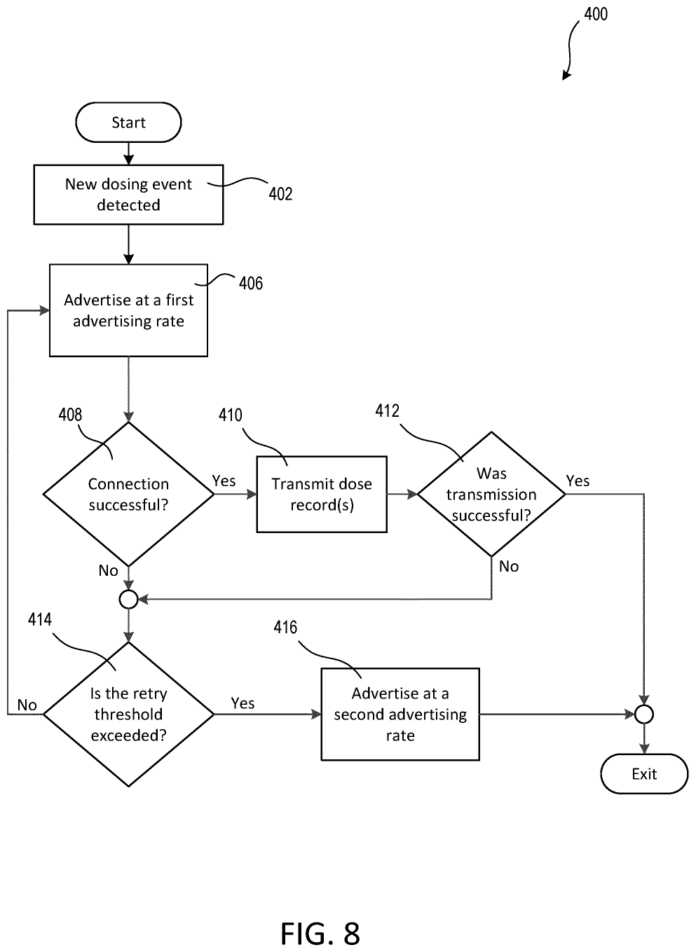

FIG. 8 illustrates an example procedure 400 for an inhalation device (e.g., such as the inhalation device 100 shown in FIGS. 1-4 and 5A-5D) to transmit one or more dose records to an external device (e.g., such as the external device 904 shown in FIG. 11 and/or the external device 1100 shown in FIG. 13). Although described with reference to the external device, the procedure 400 may be used by the inhalation device to connect with and send one or more dose records to another device. The inhalation device may comprise an electronics module (e.g., such as the electronics module 120 shown in FIGS. 2-4 and 5A-5D). The external device may be a mobile device, a personal computer, a smart hub, etc. A controller (e.g., processor) of the electronics module of the inhalation device may perform the procedure 400 in response to the storage of a new dosing record into memory of the electronics module. The electronics module may be in the sleep state or the active state at 302, when the procedure 400 begins.

The inhalation device (e.g., the electronics module) may determine, at 402, that a new dosing event has been detected (e.g., and a new dose record is stored in memory of the inhalation device). For example, as described herein, the electronics module may generate a dose record in response to a dosing event (e.g., detection of a dosing event). The dosing event may, for example, include actuation of a switch of the inhalation device (e.g., such as switch 130 shown in FIGS. 1 and 5A-5D) and/or receipt of measurements by a sensor of the inhalation device (e.g., such as sensor system 128 shown in FIG. 3 and/or sensor 1027 shown in FIG. 12). For example, the processor of the electronics module (e.g., such as processor 1026 shown in FIG. 12) may receive pressure measurements from the sensor that are indicative of an inhalation. The electronics module may determine that a dosing event occurred when the pressure measurements are greater than a threshold pressure. The threshold pressure may be indicative of a user's inhalation via the mouthpiece of the inhaler. The electronics module may be configured to store the generated dose record in the memory of the inhalation device.

At 406, the electronics module may cause the communication circuit to transmit, at 406, advertising data at a first advertising rate. As described herein, the electronics module may include a communication circuit (e.g., such as the communication circuit 1028 shown in FIG. 12), which may be a wireless communication circuit. For example, the communication circuit may be configured to communicate using one or more Bluetooth communication protocols. The communication circuit may be configured to enter a connected state with the external device when the communication circuit is “paired” with the external device.

The first advertising rate may be referred to as a fast advertising rate. For example, the first advertising rate may be once every 100 ms. The communication circuit may transmit the advertising data at the first advertising rate for an advertising period. The advertising period may be 60 seconds. In some examples, the detection of the dosing event (e.g., the actuation of the switch and/or reception of sensor data) may initiate a timer (e.g., an advertising period timer). In some examples, the timer may be configured to expire 180 seconds after the detecting of the dosing event, and one or more advertising periods may be used by the electronics module in the 180 seconds after actuation of the switch. Further, in addition to starting a timer, the electronics module may also set a retry value at 408. As described in more detail below, the electronics module may use the retry value (NRETRY) to determine the number of advertising periods that the electronics module has transmitted advertising data at the first advertising rate after the recording of a dose record at 402.

The advertising data may include any of the information described herein. For example, the advertising data may include a dose record indicator, a total number of dose records stored in the memory of the electronics module, an indication of a present dose count of the inhalation device, and/or a data available indication. As described herein, the dose record indicator may indicate that one or more dose records are stored in the memory of the electronics module (e.g., the dose record indicator may indicate the most recently stored dose record(s) in memory).

At 408, electronics module may determine whether a connection between the communication circuit and the external device was successful. For example, the electronics module may determine, at 408, whether the communication circuit is in a connected state with the external device. If the electronics module determines that the communication circuit is in the connected state with the external device (e.g., a connection was successful) at 408, the electronics module may cause the communication circuit to transmit, at 410, the one or more dose records to the external device. For example, the external device may transmit a request to the electronics module for one or more dosing records, for instance, based on the dose record indicator provided in the advertising data. In response, the electronics module may transmit those requested dose records to the external device. At 412, the electronics module may determine whether the transmission of the one or more dose records was successful at 410. When the electronics module determines that the transmission of the one or more dose records was successful at 410, the procedure 400 may end. Further, in some examples, after determining that the transmission of the one or more dose records was successful, the electronics module may reset the retry value (NRETRY).

However, if the electronics module determines that the communication circuit has not entered a connected state with the external device (e.g., a connection was successful) at 408 and/or if the electronics module determines that the transmission of the one or more dose records was not successful at 412, the procedure 400 may proceed to 414 and the electronics module may determine whether a connection retry threshold (NTH) has been exceeded. The transmission of the dose record(s) may not be successful due to a variety of causes and circumstances, such as, a loss of connection between the electronics module and external device. This may occur when the devices move apart and outside of a communication range with one another. Alternatively or additionally, radio interference could cause the electronics module to lose its connection with the external device and cause the transmission to be unsuccessful.

The electronics module may determine whether the connection retry threshold (NTH) has been exceeded by comparing the retry value (NRETRY) to the connection retry threshold (NTH). The electronics module may adjust (e.g., increase or decrease) the retry value in response to a failed data transmission (at 412) and/or in response to the expiration of the advertising period timer. For instance, if the advertising period timer expires, the electronics module may reset the timer and increase the retry value (NRETRY). As such, the connection retry threshold (NTH) may represent a maximum number of consecutive advertising periods that the electronics module may execute at the first advertising rate. The connection retry threshold (NTH) may be determined based on a combined time of each of the consecutive advertising periods at the first advertising rate. As an example, the connection retry threshold (NTH) may be three and the advertising period may be 60 seconds. In such instances, electronics module may be configured to transmit advertising data at the first advertising rate for three advertising periods (e.g., a combined time of 180 seconds, without any connection events and/or failed data transfers).

If the electronics module determines that the connection retry threshold (N_{TH}) has not been exceeded 414, the electronics module may return to 406 and cause the communication circuit to continue to transmit advertising data at the first advertising rate. However, if the electronics module determines that the connection retry threshold (N_{TH}) has been exceeded 414, the electronics module may cause the communication circuit to transmit, at 416, advertising data at a second advertising rate before exiting the procedure 400. The second advertising rate may be slower (e.g., less frequent) than the first advertising rate, and as such, the second advertising rate may be referred to as a slow advertising rate. For example, the second advertising rate may be once every 10 seconds. The electronics module may use the slower advertising rate to save resources and increase battery life, while still maintaining connection and measurement capabilities. The electronics module may continue to use the slow advertising rate, for example, until another dosing event occurs and/or until the electronics module successfully transmits one or more dosing events (e.g., all new dosing events).

As such, the electronics module may perform the procedure 400 to optimize advertising and/or save resources (e.g., battery life). For example, the inhalation device may be in close proximity to the external device following (e.g., immediately following) a dosing event and/or a failed data transmission, and as such, there may be an increased likelihood that the inhalation device remains in close proximity to the external device after these events. Thus, the electronics module is configured to transmit advertising data at a faster rate following (e.g., immediately following) the dosing event and/or a failed data transmission, and at a less frequent advertising rate thereafter. Note that the use of the retry value and retry threshold together with multiple advertising periods offers advantages compared with simply setting a single, longer advertising period. A single long advertising period may increase the chances of establishing communication with an external device. However, if data transmission then fails, it will take longer to re-establish communications, because the wireless communication circuit of the electronics module will then be using the second advertising rate. In contrast, according to the present example, the wireless communication circuit would use the first advertising rate for a second (short) advertising period. This increases the chances that communication can be re-established as quickly as possible, while avoiding excessive power consumption. Indeed, the overall amount of time that the electronics module is permitted to use the first (faster) advertising rate need not be increased. Instead, the total period for which it is desired to operate with the first advertising rate can be split into a number of shorter advertising periods.

In some examples, the inhalation device may include a rechargeable battery. In such instances, the inhalation device may be configured to reduce battery power when the energy level (e.g., power level, charge level, voltage, etc.) of the battery drops below a threshold. For instance, the inhalation device may be configured to prevent and/or reduce the transmission of dose records to the external device, for example, when the electronics module is in the low power active state and/or the energy level of the battery is below the threshold. For example, if the energy level of the battery is below the threshold, the inhalation device may determine to reduce or not transmit advertising data and/or one or more dose records, which in turn may optimize battery life and ensure that adequate battery life is available to perform further measurements related to dosing events. As such, when the energy level of the battery is below the threshold, the inhalation device may continue to record new dose records, but may stop or limit the amount of power is used to communicate with an external device (e.g., until the battery is recharged). In this way, the inhalation device can prioritize its most important functions-making measurements related to dosing events and storing new dose records. The inventors have recognized that, provided a complete set of new dose records is recorded, a delay in communicating those records to an external device may be tolerable to the user. Additionally, the refusal of the inhalation device to communicate with the external device may act as a prompt to the user, making them aware that the battery of the inhalation device is running low and providing them with an additional incentive to recharge it-namely, in order to restore communication between the inhalation device and the external device and enable transfer of the records to the external device. When communication is restored, new dose records recorded since the last successful transfer may be transferred in a batch.

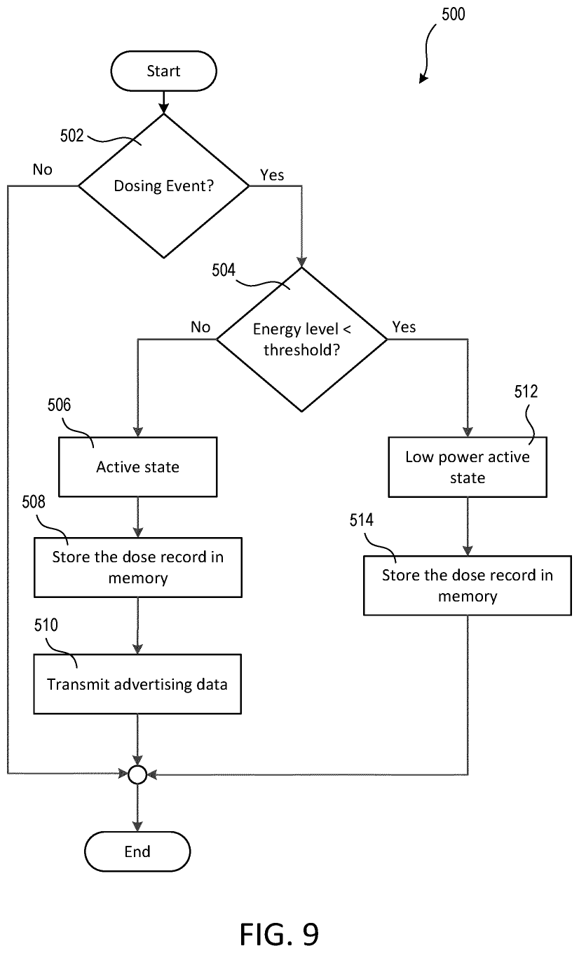

FIG. 9 illustrates an example procedure 500 for an inhalation device (e.g., such as the inhalation device 100 shown in FIGS. 1-4 and 5A-5D) to select an advertising rate based on a energy level of a battery of the inhalation device (e.g., such as the battery 126 shown in FIG. 4 and/or the power supply 1029 shown in FIG. 12). The inhalation device may advertise data to and attempt to connect with an external device (e.g., such as the external device 904 shown in FIG. 11). The inhalation device may comprise an electronics module (e.g., such as the electronics module 120 shown in FIGS. 2-4 and 5A-5D). The external device may be a mobile device, a personal computer, a smart hub, etc. A controller (e.g., processor) of the electronics module of the inhalation device may perform the procedure 500 periodically and/or in response to a dosing event occurring at the inhalation device. The electronics module may be in the sleep state at 502, when the procedure 500 begins.

At 502, the electronics module may determine whether a dosing event occurred at the inhalation device (e.g., and a new dose record is stored in memory of the inhalation device). For example, as described herein, the electronics module may generate a dose record in response to a dosing event (e.g., detection of a dosing event). The dosing event may, for example, include actuation of a switch of the inhalation device (e.g., such as switch 130 shown in FIGS. 1 and 5A-5D) and/or receipt of measurements by a sensor of the inhalation device (e.g., such as sensor system 128 shown in FIG. 3 and/or sensor 1027 shown in FIG. 12). For example, the processor of the electronics module (e.g., such as processor 1026 shown in FIG. 12) may receive pressure measurements from the sensor that are indicative of an inhalation. The electronics module may determine that a dosing event occurred when the pressure measurements are greater than a threshold pressure. The threshold pressure may be indicative of a user's inhalation via the mouthpiece of the inhaler. The electronics module may be configured to store the generated dose record in the memory of the inhalation device.

If the electronics module determines that a dosing event has not occurred, the procedure 500 may end. However, if the electronics module determines that a dosing event has occurred, the electronics module may determine, at 504, whether an energy level of the battery is below a predetermined threshold. For example, the electronics module may determine the energy level of the battery and compare the energy level of the battery to the predetermined threshold. For instance, the electronics module may receive a battery voltage VBATT that indicates the voltage of the battery, and compare the battery voltage to one or more battery voltage thresholds VTH. Further, in some examples, the electronics module may enter an active state or a low power active state in response to the occurrence of a dosing event (e.g., actuation of the switch), for example, using the procedure 500. For instance, the electronics module may be configured to be in one of a plurality of power states, such as an off state, a sleep state, an active state, and/or a low power active state. The electronics module may be configured to use more power when in the active state than when in the low power active state, may be configured to use more power when in the low power active state than when in the sleep state, and may be configured to use more power when in the sleep state than when in the off state.

If the electronics module determines that the energy level of the battery is not less than a threshold at 504, the electronics module may enter the active state at 506. For instance, the electronics module may be in a sleep state prior to the detection of the dosing event at 502, and may transition to the active state in response to a determination that the energy level of the battery is not less than the threshold at 504. When the electronics module is in the active state, the electronics module may, inter alia, generate one or more dose records and transmit advertising data to the external device (e.g., in an attempt to connect and transmit the dosing event to the external device). For instance, the electronics module may generate the dose record and store the dose record in memory of the electronics module at 508, and the electronics module may transmit advertising data to the external device at 510, before the procedure 500 may exit. In some examples, the electronics module may transmit advertising data at a first advertising rate (e.g., a fast advertising rate) to the external device at 510 (e.g., as described with reference to procedure 400). Further, the advertising data may include a dose record indicator (e.g., as described with reference to the procedure 300).

If the electronics module determines that the energy level of the battery is less than the threshold at 504, the electronics module may enter the low power active state at 512. For instance, the electronics module may be in a sleep state prior to the detection of the dosing event at 502, and may transition to the low power active state in response to a determination that the energy level of the battery is less than the threshold at 504. When the electronics module is in the low power active state, the processor may be configured to record a dose record based on the dosing event (e.g., based on the actuation of the switch and/or received measurements) at 514. For instance, when in the low power active state, the electronics module may be configured to power the sensor system and receive measurements from the sensor system. The electronics module may generate a dose record in response to a dosing event (e.g., detection of a dosing event) at 514, and the procedure 500 may exit. Notably, in some examples, the electronics module may be configured to generate dose records when in the low power active state, but the electronics module may not be configured to transmit advertising data. For instance, when in the low power active state, the electronics module may be configured to not power the communication circuit and not transmit data to the external device. This may allow the inhalation device to save battery power and still record new dose records when the battery level is below a threshold. Such examples may be particularly valuable in situations where the battery of the inhalation device is rechargeable. As such, upon the battery of the inhalation device being recharged (e.g., and the energy level exceeding the threshold), the inhalation device may be configured to enter the active state and transmit stored dosing records to the external device.

Further, in some examples, the electronics module may be configured to cause the communication circuit to transmit the advertising data to the external device at a second advertising rate when in the low power active state. The second advertising rate may be slower than the first advertising rate. For example, the second advertising rate may be a slow advertising rate, such as once every 10 seconds.

Accordingly, using the procedure 500, the electronics module may be configured to prevent the communication circuit from transmitting the dose record to the external device, for example, when the electronics module is in the low power active state and/or the energy level of the battery is below the threshold. The electronics module may determine whether to transmit advertising data and/or one or more dose records based on the energy level of the battery, for example, to optimize battery life, ensure that adequate battery life is available to perform future measurements and/or record future dose records. Accordingly, the use of the procedure 500 and the low power active state may prevent or reduce the transmission of data from the electronics module when the energy level of the battery is below the threshold in order to save resources and increase battery life.

Further, although described as using a single threshold, in some examples, the electronics module may be configured with multiple power thresholds, with each power threshold being associated with a different level of power usage at the electronics module. For instance, the electronics module may be configured with a first power threshold, above which the electronics module is configured to enter the active state. Further, if the energy level of the battery of the electronics module is below the first power threshold and greater than a second power threshold, the electronics module may be configured to enter a first low power active state. In the first low power active state, the electronics module may be configured to generate dose records and transmit advertising data at a slow advertising rate, or the electronics module may be configured to generate dose records and transmit advertising data at a fast advertising rate but for an advertising period that is less than the advertising period that is used when in the active state. Using the slow advertising rate and/or the fast advertising rate for shorter amount of time may save resources and increase battery life of the electronics module and the external device while maintaining connection and measurement capabilities. Further, if the energy level of the battery of the electronics module is below the second power threshold, the electronics module may be configured to enter a second low power active state. When in the second low power active state, the electronics module may be configured to generate dose records, but not transmit advertising data or power the communication circuit (e.g., similar to the low power state described at 512). As such, the electronics module may be configured with one or more intermediate low power states, in which the electronics module is configured to use progressively less power than when in the active state.

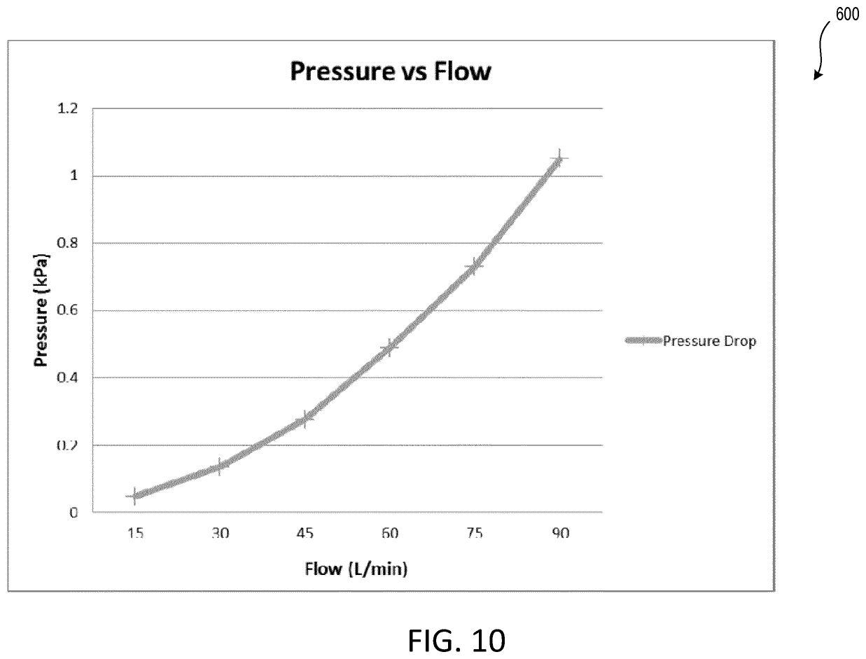

FIG. 10 shows an example of airflow rates based on various pressure measurements calculated by a sensor system of an electronic module of an inhalation device, such as the inhalation device 100. It will be appreciated that the graph 600 of airflow rates and pressure drops shown in FIG. 10 are merely examples, and may vary based on the size, shape, and design of the inhalation device 100 and its internal components.

FIG. 11 is a diagram of an example system 900 including an inhalation device 902, an external device 904 (e.g., such as a mobile device, a personal computer, a smart hub, etc.), a public and/or private network 906 (e.g., the Internet, a cloud network), a health care provider 908, and a third party 910 (e.g., friends, family, pharmaceutical manufacturer, etc.). The mobile device 904 may include a smart phone (e.g., an iPhone® smart phone, an Android® smart phone, or a Blackberry® smart phone), a personal computer, a laptop, a wireless-capable media device (e.g., MP3 player, gaming device, television, a media streaming devices (e.g., the Amazon Fire TV, Nexus Player, etc.), etc.), a tablet device (e.g., an iPad® hand-held computing device), a Wi-Fi or wireless-communication-capable television, or any other suitable Internet-Protocol-enabled device. For example, the mobile device 904 may be configured to transmit and/or receive RF signals via a Wi-Fi communication link, a Wi-MAX communications link, a Bluetooth® or Bluetooth Smart communications link, a near field communication (NFC) link, a cellular communications link, a television white space (TVWS) communication link, or any combination thereof. The mobile device 904 may transfer data through the public and/or private network 906 to the health care provider 908 and/or one or more third parties 910 (e.g., friends, family, pharmaceutical company, etc.).

The inhalation device 902 may be an example of the inhalation device 100. The inhalation device 902 may include a communication circuit, such as a Bluetooth radio, for transferring data to the mobile device 904. The data may include the signals generated by the switch 130, the pressure measurement readings taken by the sensory system and/or the airflow metrics computed by the controller 127 of the electronics module. The inhalation device 902 may receive data from the mobile device 904, such as, for example, program instructions, operating system changes, dosage information, alerts or notifications, acknowledgments, etc.

The mobile device 904 may process and analyze the data to determine the usage parameters associated with the inhalation device 902. For example, the mobile device 904 may process the data to identify no inhalation events, low inhalations events, good inhalation events, excessive inhalation events and/or exhalation events. The mobile device 904 may also process the data to identify underuse events, overuse events and optimal use events. The mobile device 904 may further process the data to estimate the number of doses delivered and/or remaining and to identify error conditions, such as those associated with a timestamp error flag. The mobile device 904 may include a display and software for visually presenting the usage parameters through a GUI on the display.

Further, in some examples, the inhalation device 900 may include an actuator to initiate a pairing process with the mobile device 904. However, the inhalation device 900 may include other means for facilitating the pairing process. For example, the top cap of the inhalation device 900 may include a Quick Response (QR) code. The mobile device 904 may include a camera and software application for accessing the camera and reading the QR code. The QR code may include a BLE passkey that is unique to the inhalation device 900. Upon reading or scanning the QR code using the camera, the software application may receive the BLE passkey associated with the device 900 and complete an authentication process, thereby enabling it to communicate with the electronics module using the BLE passkey. If the communications session is subsequently lost because, for example, the inhalation device 900 moves out of range, the mobile device 904 may be configured to use the BLE passkey to automatically pair with the electronics module without using the QR code when the inhalation device 900 is back within range.

The external device 902 may include a smart phone (e.g., an iPhone® smart phone, an Android® smart phone, or a Blackberry® smart phone), a personal computer, a laptop, a wireless-capable media device (e.g., MP3 player, gaming device, television, a media streaming devices (e.g., the Amazon Fire TV, Nexus Player, etc.), etc.), a tablet device (e.g., an iPad® hand-held computing device), a Wi-Fi or wireless-communication-capable television, or any other suitable Internet-Protocol-enabled device. For example, the external device 902 may be configured to transmit and/or receive RF signals via a Wi-Fi communication link, a Wi-MAX communications link, a Bluetooth® or Bluetooth Smart communications link, a near field communication (NFC) link, a cellular communications link, a television white space (TVWS) communication link, or any combination thereof. The external device 902 may transfer data through the public and/or private network 904 to the DHP 906 using, for example, a dedicated API. For example, the external device 902 may send usage data relating to one or more inhalation devices 900 to the DHP 906.

The external device 902 may comprise a mobile application that is associated with the inhalation device 900. The external device 902 may be configured to process and/or analyze the data received from the inhalation device 900 to determine the usage parameters associated with the inhalation device 100. For instance, in some examples, the data stored in the memory of the electronics module of the inhalation device 900 (e.g., the signals generated by the switch 170, the measurement readings taken by the sensor system 1027, the parameters computed by the controller 127 of the electronics module 120, one or more recorded events, and/or the associated timestamps) may be transmitted to the external device 902, and the external device 902 may process and analyze the data to determine the usage parameters and/or categorize the events associated with the inhalation device 900. Accordingly, in some examples, the external device 902 may receive the measurement data from the inhalation device 900, and the external device 902 may determine one or more events based on the received data, such as no inhalation events, low inhalations events, good inhalation events, excessive inhalation events and/or exhalation events. The external device 902 may also process the data to identify underuse events, overuse events and optimal use events. The external device 902 may further process the data to estimate the number of doses delivered and/or remaining and to identify error conditions, such as those associated with a timestamp error flag. As such, some of the processing that could be performed at the inhalation device 902 may be offloaded to the external device 902.

In some examples, the inhalation device 900 and/or the external device 902 may record (e.g., store) all the data associated with a single usage or inhalation event of the inhalation device 900, for example, based on the timestamps associated with the data (e.g., all timestamps that are within a particular time of one another may be categorized together). For instance, the inhalation device 900 and/or the external device 902 may record an inhalation event that includes the mouthpiece cover opening event, the canister actuation event, any errors events, the one or more signals from the sensor indicative of the user's inhalation, and/or the associated timestamps. The inhalation device 900 and/or the external device 902 may send the inhalation event to a remote server, such as the DHP 906 (e.g., send the mouthpiece cover opening event, the canister actuation event, any errors events, the one or more signals from the sensor indicative of the user's inhalation, and/or the associated timestamps as an inhalation event to the DHP 906).

The inhalation device 900 and/or the external device 902 (e.g., via a mobile application residing on the external device) may be configured to provide a notification to the user based on the user's usage of the inhalation device 900. For example, a mobile application residing on the external device may generate feedback for the user based on data received from the electronics module 120. For example, the mobile application may generate daily, weekly, or monthly report, provide confirmation of error events or notifications, provide instructive feedback to the user, and/or the like, where the reports, notifications, and/or feedback include the usage parameters, events, and/or measurements associated with the inhalation device 900. The feedback and/or notification may, for example, be the illumination of an LED, the generation of an audible output via a speaker of the inhalation device 900 or the external device 902, the presentation of a message via the display of the external device 902, the presentation of an error video or the instructions for use via the display of the external device 902, by sending a text, email, or instant message to the external device 902, etc.

Further, in some examples, the external device 902 may include a display and software for visually presenting the usage parameters through a GUI on the display. For instance, the external device 902 may be configured to cause the display to present a notification indicating any combination of the events, the measurements, and/or the associated time stamps related to one or more inhalation devices 900. For example, the external device 902 may be configured to generate a GUI that presents any data associated with an inhalation event (e.g., any combination of the mouthpiece cover opening event, the canister actuation event, any errors events, the one or more signals from the sensor indicative of the user's inhalation, and/or the associated timestamps).

The inhalation device 900 and/or the external device 902 may be configured to generate one or more error events based on the data recorded by the electronics module of the inhalation device 900. For example, the inhalation device 900 and/or the external device 902 may be configured to generate an exhalation event when the one or more signals from the sensor indicate an exhalation occurred (e.g., the one or more signals from the sensor indicate a positive pressure change, for instance, when the sensor system comprises a pressure sensor). The inhalation device 900 and/or the external device 902 may be configured to generate an error event (e.g., a mishandling event) in response to the switch contact contacting the second contact pad and the third contact pad of the electronics module and the one or more signals from the sensor indicating measurements below a threshold (e.g., the one or more signals from the sensor indicating pressure measurements below a threshold that is indicative of no inhalation, such as pressure measurements that correspond to a flow rate that is less than 20 L/min). For example, the inhalation device 900 and/or the external device 902 may be configured to generate an error event (e.g., a mishandling event) based on the electronics module recording a canister actuation event but the one or more signals from the sensor indicating measurements below the threshold. Further, in some examples, the inhalation device 900 and/or the external device 902 may be configured to generate a timeout event in response to the electronics module recording a mouthpiece covert opening event, but not a subsequent canister actuation event or the recording of measurements from the sensor system that exceeds a threshold (e.g., 20 L/min) within a set time period, such as 60 seconds.

Further, the inhalation device 900 and/or the external device 902 may be configured to generate a no inhalation error event based on the electronics module of the inhalation device 900 recording a mouthpiece cover opening event and a canister actuation event without the one or more signals from the sensor indicating a user's inhalation (e.g., without the one or more signals from the sensor indicating measurements that are indicative of a user's inhalation, such as measurements that correspond to a flow rate greater than 20 L/min). Also, the inhalation device 900 and/or the external device 902 may be configured to generate a multiple inhalation error event in response to the one or more signals from the sensor system indicating that multiple inhalation occurred during and/or after a single canister actuation event (e.g., where the signals from the sensor system exceeds a threshold, such as 20 L/min, fall below the threshold for a period of time, like 1 second, and then exceed the threshold again). Finally, in some examples, the inhalation device 900 and/or the external device 902 may be configured to generate an excessive inhalation or air vent blockage error event when the one or more signals from the sensor system exceed an upper threshold, such as when the signals correspond to a flow rate that is greater than 300 or 500 L/min).

FIG. 12 is a block diagram of an example electronics module 1020 of an inhalation device (e.g., such as the inhalation device 100 shown in FIGS. 1-4 and 5A-5D, the inhalation device and/or the inhalation device 902 shown in FIG. 11). The electronics module 1020 may be an example of the electronics module 120 shown in FIGS. 2-4 and 5A-5D. The electronics module 1020 may include a control circuit 1026 (e.g., a processor), a sensor system 1027, a communication circuit 1028, and a power supply 1029, such as a battery (e.g., such as the battery 126 shown in FIG. 4).

The processor 1026 may access information from, and store data in memory 1030 of the electronics module 1020. The memory 1030 may include any type of suitable memory, such as non-removable memory and/or removable memory. The non-removable memory may include random-access memory (RAM), read-only memory (ROM), a hard disk, or any other type of memory storage device. The removable memory may include a subscriber identity module (SIM) card, a memory stick, a secure digital (SD) memory card, and the like. The memory 1030 may be internal to the controller. The controller 127 may also access data from, and store data in, memory that is not physically located within the electronics module 1020, such as on a server or a smartphone.

The memory 1030 may comprise a computer-readable storage media or machine-readable storage media that maintains computer-executable instructions for performing one or more as described herein. For example, the memory 1030 may comprise computer-executable instructions or machine-readable instructions that include one or more portions of the procedures described herein. The controller 1026 of the electronics module 1120 may access the instructions from memory for being executed to cause the controller 1026 of the electronics module 1120 to operate as described herein, or to operate one or more other devices as described herein. The memory 1030 may comprise computer-executable instructions for executing configuration software. For example, the computer-executable instructions may be executed to perform, in part and/or in their entirety, one or more procedures, such as the procedures 200, 400, and/or 500 as described herein. Further, the memory 1030 may have stored thereon one or more settings and/or control parameters associated with the electronics module 1120.

The processor 1026 may include a microcontroller, a programmable logic device (PLD), a microprocessor, an application specific integrated circuit (ASIC), a field-programmable gate array (FPGA), and/or any suitable processing device or control circuit. The memory may include computer-executable instructions that, when executed by the processor 1026, cause the processor 1026 to implement the processes of the electronics module 1020 as described herein. When used herein, the terms controller 127 and processor may be used interchangeably.

The sensor system 1027 may include one or more sensors, such as one or more pressure sensors, temperature sensors, humidity sensors, acoustic sensors, optical sensors, orientation sensors, and/or the like. The pressure sensor(s) may include a barometric pressure sensor (e.g., an atmospheric pressure sensor), a differential pressure sensor, an absolute pressure sensor, and/or the like. The sensors may employ microelectromechanical systems (MEMS) and/or nanoelectromechanical systems (NEMS) technology. The pressure sensor(s) may be configured to provide an instantaneous pressure reading to the processor 1026 of the electronics module 1020 and/or aggregated pressure readings over time. The pressure sensor(s) may be configured to measure a plurality of atmospheric pressures within the inhalation device. Examples of the sensors 1027 are described in reference to US 2020/0360630 A1, the entire disclosure of which are incorporated herein by reference. Further, it should be appreciated that the processor 1026 of the electronics module 1020 may be configured to convert pressure measurements received from the pressure sensor to a flow rate based on Bernoulli's principle (e.g., the Bernoulli/Venturi effect).

The electronics module 1020 (e.g., and/or a mobile application residing on an external device) may use measurements from the sensor system 1027 to determine one or more dosing events. For example, the electronics module 1020 may be configured to compare one or more measurements from the sensor system 1027 to one or more threshold values to categorize an inhalation event as a no/low inhalation event, a fair inhalation event, a good inhalation event, an excessive inhalation event, and/or an exhalation event. For example, the electronics module may generate a good inhalation event when the measurements from the sensor system 1027 indicate a flow rate in a particular range (e.g., greater than 20 L/min, or between 200 liters per min (L/min) and 20 L/min), generate a no inhalation event when the measurements from the sensor system 1027 indicate a flow rate that is less than a threshold value (e.g., 20 L/min), and an excessive inhalation event when the measurements from the sensor system 1027 indicate a flow rate that is greater than an upper threshold (e.g., greater than 200 L/min). Further, although described primarily in terms of flow rate, the measurements calculated by the sensor system may be used to calculate inhalation volume and/or inhalation duration, and the thresholds may be inhalation volume thresholds and/or inhalation duration thresholds.

The communication circuit 1028 may include a transmitter and/or receiver (e.g., a transceiver), as well as additional circuitry (e.g., such as a controller 127 and/or memory). The communication circuit 1028 may include a wireless communication circuit. For example, the communication circuit 1028 may include a Bluetooth chip set (e.g., a Bluetooth Low Energy chip set), a ZigBee chipset, a Thread chipset, etc. As such, the electronics module 1020 may be configured to wirelessly provide data (e.g., the parameters determined by the processor 1026, such as pressure measurements, temperature, humidity level, orientation, etc., one or more recorded events, etc.) to an external device, including a smartphone. The external device may include software for processing the received information and for providing compliance and adherence feedback and/or any of the notifications described herein to users of the inhalation device via a graphical user interface (GUI).

The power supply 1029 may provide power to the components of the electronics module 1020. The power supply 1029 may be any suitable source for powering the electronics module 1020, such as a coin cell battery, for example. The power supply 1029 may be rechargeable or non-rechargeable. The power supply 1029 may be secured to the electronics module 1020 such that the power supply 1029 maintains continuous contact with and/or is in electrical connection with the components of a PCB of the electronics module 1020. The power supply 1029 may have a battery capacity that may affect the life of the power supply 1029. As will be further discussed below, the distribution of power from the power supply 1029 to the one or more components of the electronics module 1020 may be managed to ensure the power supply 1029 can power the electronics module 1020 over the useful life of the inhalation device 100 and/or the medication contained therein.

The electronics module 1020 may have a plurality of power states, each with respective power consumption levels. For example, the electronics module 1020 may be configured to operate in a system off state, a sleep state, a low power active state, and/or an active state. Each of the power states may be defined by different power consumption levels. For example, the electronics module 1020 may be configured to operate in a system off state, a sleep state, a low power active state, and/or an active state. The electronics module 1020 consumes the least amount of power while in the off state (e.g., no power or just enough to run a clock and/or monitor one or more processor pins in electrical communication with one or more contact pads), consumes more power in the sleep state than the off state (e.g., to drive the memory, the communication circuit, and/or a timer or clock), and consumes more power in the low power active state than in the sleep or off states, and consumes more power in the active state than in the low power active, sleep, or off states (e.g., to drive the processor 1026, the sensor system 1027, the communication circuit 1028, potentially in a faster advertising mode than the sleep state, and/or a timer or clock). Examples of the power states of an inhalation device, such as the inhalation device, are described in US 2018/0140786 A1, the entire disclosure of which is incorporated herein by reference.

While the electronics module 1020 is in the active state, the electronics module 1020 may operate in one or more modes, such as a measurement mode, a data storage/data processing mode, an advertising mode, and/or a connected mode. It should be appreciated that the electronics module 1020 may operate in multiple modes at one time (e.g., the modes may overlap).

In the measurement mode, the controller 127 of the electronics module 1020 may power on the sensor system 1027. The processor 1026 may cause the sensor system 1027 to take pressure measurement readings, temperature readings, humidity readings, orientation readings, etc. for a predetermined time period (e.g., up to 60 seconds) and/or until a mouthpiece cover of the inhalation device (e.g., such as the mouthpiece cover 108 shown in FIGS. 1-3 and 5A-5D) is closed or no changes in pressure are detected. The processor 1026 may turn off one or more components of the electronics module 1020 while the sensor system 1027 is capturing readings to further conserve power. The sensor system 1027 may sample the readings at any suitable rate. For example, the sensor system 1027 may have a sample rate of 100 Hz and thus a cycle time of 10 milliseconds. The sensor system 1027 may generate a measurement complete interrupt after the measurement cycle is complete. The interrupt may wake the processor 1026 or cause it to turn on one or more components of the electronics module 1020. For example, after or while the sensor system 1027 is sampling one or more pressure measurements, temperature readings, humidity readings, orientation readings, etc., the processor 1026 may process and/or store the data and, if measurements are complete, power off the sensor system 1027.

In the data storage/data processing mode, the processor 1026 may power on at least a portion of the memory within the electronics module 1020. The processor 1026 may process the readings from the sensor system 1027 to compute, estimate, calculate or otherwise determine parameters (e.g., usage and/or storage conditions) and store the parameters in memory. The processor 1026 may also compare the readings and/or parameters to one or more thresholds or ranges to assess how the inhalation device is being used and/or the conditions under which the inhalation device is being used. Depending on the results of the comparison, the processor 1026 may drive one or more indicators to provide feedback to the user of the inhalation device. As noted above, the electronics module 1020 may operate in the measurement mode and the data storage/data processing mode simultaneously. After determining one or more parameters (e.g., usage and/or storage conditions) from the readings of the sensor system 1027, the processor 1026 may transmit the parameters and/or associated timestamps (e.g., based on the internal counter) to the external device when in the connected mode.

In the connected mode, the communication circuit 1028 may be powered on and the electronics module 1020 may be “paired” with an external device, such as a smartphone. The processor 1026 may retrieve data from the memory and wirelessly transmit the data to the external device. The processor 1026 may retrieve and transmit all of the data currently stored in the memory. The processor 1026 may also retrieve and transmit a portion of the data currently stored in the memory. For example, the processor 1026 may be able to determine which portions have already been transmitted to the external device and then transmit the portion(s) that have not been previously transmitted. Alternatively, the external device may request specific data from the processor 1026, such as any data that has been collected by the electronics module 1020 after a particular time or after the last transmission to the external device. The processor 1026 may retrieve the specific data, if any, from the memory and transmit the specific data to the external device.

Further, when connected with the external device, the electronics module 1020 may be configured to transmit Bluetooth special interest group (SIG) characteristics for managing access to data stored in the module 1020. The Bluetooth SIG characteristics may include one or more of a manufacturer name of the inhalation device, a serial number of the inhalation device, a hardware revision number of the inhalation device, and/or a software revision number of the inhalation device. When connected with the external device, the electronics module 1020 may retrieve data from memory and transmit the data to the external device.

The electronics module 1020 may include a mouthpiece cover position sensor 1022 and a canister position sensor 1024. The mouthpiece cover position sensor 1022 may be configured to sense the position of the mouthpiece cover (e.g., open or closed), and the canister position sensor 1024 may be configured to sense the position of a medication canister (e.g., such as the medication reservoir 110 shown in FIG. 2), for example, along a longitudinal axis within a main housing (e.g., such as the main housing 104 shown in FIGS. 1-3 and 5A-5D) such as in a first, second, or third position. In some examples, the mouthpiece cover position sensor 1022 may be a contact pad, and the canister position sensor 1024 may include one or more contact pads. As such, the processor 1026 may be configured to determine the position of the mouthpiece cover and/or determine when the position of the mouthpiece cover changes based on feedback from the mouthpiece cover position sensor 1022. Further, the processor 1026 may be configured to determine the position of the medication canister and/or determine when the position of the medication canister changes based on feedback from the canister position sensor 1024. The processor 1026 may be configured to timestamp and/or transmit data indicating the position of the mouthpiece cover and/or the medication canister to the external device.

The signals generated by a switch contact contacting the contact pads and/or the measurement readings taken by the sensory system 1027 may be timestamped and stored in memory of the electronics module 1020. The foregoing parameters may be indicative of various usage and/or storage conditions associated with the inhalation device. For example, as movement of a movable inner housing causes the switch contact to contact one or more of the contact pads, the processor 1026 may use the signals from the contact pads to record and timestamp each transition. Further, since the signals from the contact pads may correlate to the position of the mouthpiece cover (e.g., open or closed), the processor 1026 may be able to detect and track the position of the mouthpiece cover and/or medicament canister over time. It will be appreciated that the processor 1026 may be able to sense and track the status of the mouthpiece cover without interfering with the delivery of medication through the flow pathway of the inhalation device.

FIG. 13 illustrates a block diagram of an example computing device 1100 (e.g., external device). The computing device 1100 may include a personal computer, such as a laptop or desktop computer, a tablet device, a cellular phone or smartphone, a server, or another type of computing device. The computing device 1100 may include a processor 1102, a communication interface 1104, a memory 1106, a display 1108, input devices 1110, output devices 1112, and/or a GPS circuit 1114. The computing device 1100 may include additional, different, or fewer components.

The processor 1102 may include one or more general purpose processors, special purpose processors, conventional processors, digital signal processors (DSPs), microprocessors, integrated circuits, a programmable logic device (PLD), application specific integrated circuits (ASICs), or the like. The processor 1102 may perform signal coding, data processing, image processing, power control, input/output processing, and/or any other functionality that enables the computing device 1100 to perform as described herein.

The processor 1102 may store information in and/or retrieve information from the memory 1106. The memory 1106 may include a non-removable memory and/or a removable memory. The non-removable memory may include random-access memory (RAM), read-only memory (ROM), a hard disk, or any other type of non-removable memory storage. The removable memory may include a subscriber identity module (SIM) card, a memory stick, a memory card, or any other type of removable memory. The memory may be local memory or remote memory external to the computing device 1100. The memory 1106 may store instructions which are executable by the processor 1102. Different information may be stored in different locations in the memory 1106.

The memory 1106 may comprise a computer-readable storage media or machine-readable storage media that maintains computer-executable instructions for performing one or more as described herein. For example, the memory 1106 may comprise computer-executable instructions or machine-readable instructions that include one or more portions of the procedures described herein. The processor 1102 of the external device 1100 may access the instructions from memory for being executed to cause the processor 1102 of the external device 1100 to operate as described herein, or to operate one or more other devices as described herein. The memory 1106 may comprise computer-executable instructions for executing configuration software. For example, the computer-executable instructions may be executed to perform, in part and/or in their entirety, one or more procedures, such as the procedure 300 as described herein. Further, the memory 1106 may have stored thereon one or more settings and/or control parameters associated with the external device 1100.

The processor 1102 that may communicate with other devices via the communication device 1104. The communication device 1104 may transmit and/or receive information over the network 1116, which may include one or more other computing devices. The communication device 1104 may perform wireless and/or wired communications. The communication device 1104 may include a receiver, transmitter, transceiver, or other device capable of performing wireless communications via an antenna. The communication device 1104 may be capable of communicating via one or more protocols, such as a cellular communication protocol, a Wi-Fi communication protocol, Bluetooth®, a near field communication (NFC) protocol, an internet protocol, another proprietary protocol, or any other radio frequency (RF) or communications protocol. The computing device 1100 may include one or more communication devices 1104.

The processor 1102 may be in communication with a display 1108 for providing information to a user. The information may be provided via a user interface on the display 1108. The information may be provided as an image generated on the display 1108. The display 1108 and the processor 1102 may be in two-way communication, as the display 1108 may include a touch-screen device capable of receiving information from a user and providing such information to the processor 1102. The processor 1102 may be configured to generate, on the display 1108, an indication of any event and/or dose record generated by and communication from the inhalation device to the external device 1100.

The processor 1102 may be in communication with a GPS circuit 1114 for receiving geospatial information. The processor 1102 may be capable of determining the GPS coordinates of the wireless communication device 1100 based on the geospatial information received from the GPS circuit 1114. The geospatial information may be communicated to one or more other communication devices to identify the location of the computing device 1100.

The processor 1102 may be in communication with input devices 1110 and/or output devices 1112. The input devices 1110 may include a camera, a microphone, a keyboard or other buttons or keys, and/or other types of input devices for sending information to the processor 1102. The display 1108 may be a type of input device, as the display 1108 may include touch-screen sensor capable of sending information to the processor 1102. The output devices 1112 may include speakers, indicator lights, or other output devices capable of receiving signals from the processor 1102 and providing output from the computing device 1100. The display 1108 may be a type of output device, as the display 1108 may provide images or other visual display of information received from the processor 1102.

What is claimed is:

1. A system comprising:
an inhaler comprising:
a main body having a mouthpiece; and
an electronics module comprising a processor, memory, a wireless communication circuit and one or more of a sensor or a switch;
wherein the processor of the electronics module is configured to
generate a first dose record in response to a dosing event, wherein the dosing event comprises actuation of the switch or measurements received by the sensor;
store the first dose record in memory; and
cause the wireless communication circuit to transmit advertising data, wherein the advertising data comprises a dose record indicator that indicates that one or more dose records are stored in the memory of the electronics module, wherein the one or more dose records comprises the first dose record; and
a computer-readable storage medium comprising executable instructions that, when executed by a processor of an external device, cause the processor of the external device to:
determine, based on the dose record indicator, that the first dose record has not previously been transmitted by the electronics module; and
determine to enter a connected state with the wireless communication circuit of the electronics module in response to the determination that the first dose record has not previously been transmitted by the electronics module.

2. The system of claim 1, wherein the computer-readable storage medium is further configured to cause the processor of the external device to:
determine to not enter the connected state with the wireless communication circuit of the electronics module in response to a determination that the advertising data indicates that the one or more dose records stored in the memory of the electronics module were previously transmitted by the electronics module.

3. The system of claim 1, wherein the computer-readable storage medium is further configured to cause the processor of the external device to:
determine to not enter the connected state with the wireless communication circuit of the electronics module in response to the dose record indicator missing from the advertising data.

4. The system of claim 1, wherein the dose record indicator comprises an indication of a total number of dose records stored in the memory.

5. The system of claim 1, wherein the dose record indicator comprises an indication of a present dose count of the inhaler.

6. The system of claim 1, wherein the dose record indicator comprises an indication of the number of dosing events stored in the memory since the last time the inhaler entered a connected state with an external device.

7. The system of claim 1, wherein the processor of the electronics module is configured to receive a scan request from the external device, and cause the wireless communication circuit to transmit a scan response message, wherein the scan response message comprises the last two dose records generated by the processor of the electronics module.

8. The system of claim 7, wherein the scan response message comprises any one or any combination of two or more of: an inhalation start time, an inhalation duration, an inhalation peak, a time-to-peak, or an inhalation volume.

9. The system of claim 8, wherein the scan response message further comprises a switch actuation start time or a switch actuation close time.

10. The system of claim 7, wherein the computer-readable storage medium is further configured to cause the processor of the external device to:
send the scan request to the wireless communication circuit of the electronics module in response to the determination that the one or more dose records have not been previously transmitted by the electronics module; and
determine to enter a connected state with the wireless communication circuit of the electronics module in response to a determination that the last two dose records included in the scan response message have not been previously transmitted by the electronics module.

11. The system of claim 1, wherein the advertising data comprises a data available indication that indicates that data is available from the inhalation device and is ready for wireless download.

12. The system of claim 1, wherein the wireless communication circuit comprises a Bluetooth communication circuit.

13. The system of claim 1, wherein the processor of the electronics module is configured to cause the communication circuit to broadcast the advertising data at an advertising rate while in an advertising mode.

14. The system of claim 1, wherein the inhaler further comprises a mouthpiece cover, and where movement of the mouthpiece cover from a closed position to an open position is configured to cause the switch to be actuated.

15. The system of claim 1, wherein the processor of the electronics module is configured to generate the one or more dose records in response to measurements received from the sensor exceeding a threshold indicative of a user's inhalation via the mouthpiece of the inhaler.

16. The system of claim 1, wherein the computer-readable storage medium is further configured to cause the processor of the external device to:
determine that the first dose record has not been previously transmitted by the electronics module to the external device when the first dose record has not been transmitted to the external device or any other device.

17. The system of claim 1, wherein the computer-readable storage medium is further configured to cause the processor of the external device to:
determine that the first dose record has not been previously transmitted by the electronics module to the external device when the first dose record has been transmitted to another external device.

18. The system of claim 1, wherein the computer-readable storage medium is further configured to cause the processor of the external device to receive the first dose record from a server; and
determine to not enter the connected state with the wireless communication circuit of the electronics module in response to a determination that the first dose record indicated by the advertising data was received by the external device from the server.

19. The system of claim 1, wherein the processor of the electronics module is configured to send the first dose record to a server.

20. The system of claim 1, wherein the computer-readable storage medium is further configured to cause the processor of the external device to receive a second dose record of the inhaler from a server; and
determine to not enter the connected state with the wireless communication circuit of the electronics module in response a reception of advertising data from the inhaler that includes a second dose record indicator that indicates the second dose record.

21.-98. (canceled)
